(19) United States
(12) Patent Application Publication
(10) Pub. No.: US 2025/0302943 A1
(43) Pub. Date: Oct. 2, 2025

(54) RE-FOCUSING PROTEIN BOOSTER IMMUNIZATION COMPOSITIONS AND METHODS OF USE THEREOF

(71) Applicant: King Abdullah University of Science and Technology, Thuwal (SA)

(72) Inventors: Magnus Albert Rueping, Thuwal (SA); Jorg Eppinger, Nufahrn bei Freising (DE); Dominik Renn, Thuwal (SA); Jasdave Chahal, Woburn, MA (US); Justine McPartlan, Woburn, MA (US)

(21) Appl. No.: 18/707,713

(22) PCT Filed: Nov. 7, 2022
(86) PCT No.: PCT/IB2022/060700

§ 371 Date: May 6, 2024

Related U.S. Application Data
(60) Provisional application No. 63/276,160, filed on Nov. 5, 2021.

(51) Int. Cl. A61K 39/215; A61K 9/00; A61K 38/00; A61K 39/00; A61K 39/385; A61P 31/14; C07K 14/005; C12N 7/00

(52) U.S. Cl. CPC A61K 39/215; A61K 9/0019; A61K 39/385; A61P 31/14; C07K 14/005; C12N 7/00; A61K 38/00; A61K 2039/543; A61K 2039/55505; A61K 2039/55555; A61K 2039/575; A61K 2039/6081; C07K 2319/20; C12N 2770/20022; C12N 2770/20034

(57) ABSTRACT

Booster immunization compositions and methods of use thereof, are provided. The disclosed compositions use adjuvanted proteins as a booster vaccine targeting functionally relevant pathogen B and T cell epitopes to re-focus a patient's adaptive antibody and cytotoxic T-cell response. The pharmaceutically active ingredient of the re-focusing boost vaccines include of one or several recombinant protein molecules in a full-length or truncated yet functional (meaning the overall antigenic tertiary structure is retained) version of disease-related protein or protein domain, a smaller subunit or specific or modified epitope or combination of shorter epitopes derived from that initial prime antigen sequence. Methods for re-focusing an immune response in a subject, to augment an existing (not sufficiently protective immune response) and effectively neutralize a pathogen of interest in a mammalian host organism include administering to a subject whose immune system has been primed by a previous infection/vaccination against the pathogen.

### CROSS REFERENCE TO RELATED APPLICATIONS

This application is a National Phase application under 35 U.S.C. 371 of PCT/IB2022/060700, filed Nov. 7, 2022, which claims the benefit of and priority to U.S. Ser. No. 63/276,160, filed on Nov. 5, 2021, which is incorporated by reference herein in its entirety.

### REFERENCE TO SEQUENCE LISTING

The Sequence Listing submitted as an XML named “KAUST_2022-041-02_PCT_ST26.xml” created on Dec. 2, 2024, and having a size of 37,121 bytes is hereby incorporated by reference pursuant to 37 C.F.R. § 1.834 (c) (1).

### FIELD OF THE INVENTION

This invention is generally in the field of protein booster immunization compositions and methods of use thereof.

### BACKGROUND OF THE INVENTION

Vaccination is the administration of an antigenic material, commonly known as vaccine, to a subject in order to produce immunity to a disease or condition. Vaccination requires the establishment of a solid immune response.

The immune response that is activated by vaccination depends on the interaction of several cell types, such as T-, B- and antigen presenting cells as well as several different molecules, primarily antigens, MHC molecules, T- and B-cells receptors. A successful vaccine generates potent and long-term protection against a pathogen of interest. While a single-dose vaccine is convenient and cost-effective, in many instances a subsequent boost immunization against the pathogen is required to ensure persistent cellular and humoral immunity. The COVID-19 pandemic has promoted an interest in the development of efficient vaccines to control outbreaks and protecting populations against the disease. The advent of several severe acute SARS-COV-2 variants of concern (VOC), such as Beta (B.1.351), Delta (B.1.617.2), and Omicron (B.1.1.529) have raised worldwide concerns due to their higher transmissibility and/or pathogenicity. Specifically, Omicron represents the development of a new serotype 2, with Wuhan/Delta representing the first, wherein the prevalence duration is unknown. In addition, the VOCs have also demonstrated partial evasion of natural and vaccine-elicited neutralizing antibodies and are correlated with reduction of vaccine effectiveness. Available data suggest limited long-term durability and narrow protection in the face of the evolution and new variants of the Spike protein. Considering the risk of waning immunity after natural infection or immunization and the risk of vaccine escape by emerging variants, booster vaccines are vital.

Most COVID-19 prime-boost immunization focus on the SARS-COV-2 full length Spike protein and protection is limited to SARS-COV-2. With limited long-term durability and narrow protection in the face of a mutating Spike protein, public health officials are reporting a rise of vaccine-resistant variants and increasing cases of re-infection across the globe. The continued emergence of the variants of concern VOCs and ‘breakthrough cases’ affecting vaccinated individuals demonstrate the need for a re-focusing booster immunization.

Current approaches towards booster vaccines also focus on modified Spike antigen containing the mutations identified in Beta, Delta, and Omicron variants. While T cell response and recall of B cell memory seem more durable and may be important in protection from severe disease, data on neutralizing antibody levels suggest a rapid decline. Correspondingly, reinfections are likely to occur 3-63 month after peak antibody response with a median of 16 month [Townsend 2021], indicating a yearly boost requirement. There is still a need for more focused booster vaccine which improve the levels of neutralizing antibodies with reduced side effects on the subject.

It is an object of the present invention to provide compositions for re-focusing and boosting neutralizing antibodies against a coronavirus infection in a subject in need thereof.

It is also an object of the present invention to provide a method for refocusing and boosting neutralizing antibodies against a coronavirus infection in a subject in need thereof.

### SUMMARY OF THE INVENTION

Booster immunization compositions and methods of use thereof, are provided. The disclosed compositions use adjuvanted peptides as a booster vaccine formulations targeting functionally relevant pathogen B and T cell epitopes to re-focus a patient's adaptive antibody and cytotoxic T-cell response. In contrast to currently administered prime/boost vaccinations, which utilize the same antigen sequence for the initial vaccination (prime) and subsequent vaccination (boost), the boost compositions disclosed herein include an antigen source that is different from the antigen used at initial vaccination (prime), and will elicit an immune response and immune memory cell formation building on the pre-existing immune memory yet augmenting it with a focus on the most relevant variants by increasing the levels of neutralizing antibodies when administered to a subject, following an initial prime vaccine administration. This is hereby referred to the ‘re-focusing boost’ principle.

The pharmaceutically active ingredient of the re-focusing boost vaccines include a truncated yet functional (meaning the overall antigenic tertiary structure is retained) version of disease-related protein or protein domain, a smaller subunit or specific or modified epitope or combination of shorter epitopes derived from that initial prime antigen sequence, in combination with an adjuvant. In one preferred embodiment, the protein vaccine includes a peptide subunit, such as the receptor binding domain (RBD) of a virion surface protein for example, a betacoronavirus such as MERS-COV or SARS-COV-2. The RBD is included in the formulation as a monomer or a fusion peptide including at least two RBD sequences in tandem. The RBD is preferably be included in the formulation in tandem, including more than one RDB sequence, for example, as a dimer, trimer, tetramer or oligomer including multiple RBD units, which can be the same or different (i.e., hetero-tandem (=two different RBD's), multi genetic fusion (more than 2 RBDs) and hetero-multi genetic fusion RBD); with the individual RBD's optionally separated by a linker. In more preferred embodiments, the more than one RDB sequences in the dimer include at least two different RDB sequences (hetero-tandem) for example, RBD sequence from two different variants of the same virus. An example for a hetero-tandem RBD sequence is SEQ ID NO:16. The booster formulations disclosed herein preferably include alum as the adjuvant.

One embodiment provide refocusing boost composition, containing an effective amount of active ingredient to increase neutralizing antibodies against MERS-COV. Exemplary formulations include as the active ingredient, RDB peptides represented by SEQ ID NO: 2, 4, 6, 18, 19 or 20 or a functional variant thereof having more than 90%, 91%, 92%, 93%, 94%, 95%, 96%, 97%, 98%, or 99% identical to the amino acid sequence of SEQ ID NOs. 2, 4, 6, 18, 19 or 20. Another embodiment provides refocusing boost compositions, containing an effective amount of active ingredient to increase neutralizing antibodies against SARS-COV-2. Exemplary formulations include as the active ingredient, RDB peptides represented by SEQ ID NO: 8, 10, 12, 15, 16, 21, 22 or 23 or a functional variant thereof having more than 90%, 91%, 92%, 93%, 94%, 95%, 96%, 97%, 98%, or 99% identical to the amino acid sequence of SEQ ID NOs 8, 10, 12, 15 and 16, alone or in combination.

Also disclosed is a method of a re-focusing an immune response in a subject, by augmenting an existing, yet not sufficiently protective immune response to effectively neutralize a pathogen of interest in a mammalian host organism. An exemplary pathogen is a coronavirus (CoV), such as SARS-COV-2, MERS-COV, or HCoVs vaccine for re-focusing boost immunization. The disclosed formulations are administered to a subject in need thereof, such as a subject whose immune system has been primed by a previous infection by the coronavirus or a previous vaccination against the pathogen, preferably, previous vaccination using a vaccine wherein the antigen is delivered by administering a nucleic acid such as an mRNA, encoding an antigen such as the full length spike protein of the coronavirus.

### BRIEF DESCRIPTION OF THE DRAWINGS

FIGS. 1A-C show humoral responses against SARS-COV-2 Wuhan (FIG. 1A), SARS-COV-2 B.1.351 variant (FIG. 1B),, and MERS (FIG. 1c) RBD, induced by different alum-adjuvanted recombinant protein vaccines.

FIG. 2 shows preliminary prime-boost experiment to evaluate effective dose range of recombinant protein as a booster immunization after RNA prime. Direct mouse IgG ELISA was performed on recombinant SARS-COV-2 Wuhan RBD protein. Bar height represents the geometric mean of the individual values of each group. Dotted bar indicates immune response on RNA prime only, dashed bar indicates immune response on boost only. PBS injection served as negative control (grey). The grey values at the bottom inside the graph indicate surrogate VNT % inhibition . . .

FIG. 3 Shows the plasmid map of pATX1.

FIG. 4 is an overview of Upstream Process of Spike RBD Antigens.

FIG. 5 shows the sequence alignment of the Spike protein's RBD-regions of known human coronaviruses. Blue letters indicate the minimal RBD sequence based on structural and literature assessment. Shades refer to expressed RBD proteins: underline=first amino acid of expressed sequence, bold=last amino acid of expressed sequence, italics=sequence of peptide NVNFNFNGL (SEQ ID NO:14) T-cell epitope for mice studies with amino acids in italics present in original sequence and amino acids in italics+underline, substituted in original sequence. Note that three MERS-RBD-constructs were expressed as described in the Materials and Methods.

FIG. 6A-C show the levels of inflammatory IL-6 (FIG. 6A), FIG. 6B) CXCL1 (FIG. 6B and RANTES (FIG. 6C) following immunization with the indicated vaccines at 15 or 3 μg dose levels for the RBD protein and 5 μg dose levels for the RNA/LNP formulation. Serum collection at 6 (white bars) and 24 hrs (grey bars) after the last injection. PBS injection served as negative control. Bar height represents the geometric mean of the individual values of each group.

FIGS. 7A-C are bar graphs showing the results following direct hamster IgG ELISA performed on recombinant SARS-COV-2 Wuhan (FIG. 7A), Delta (FIG. 7B) or Omicron BA.1 (FIG. 7C) RBD protein. RNA: lipid nanoparticle containing RNA coding for full-length Beta variant spike antigen; RBDdb: 1:1 mixture of SARS-COV-2 Spike RBD protein of Beta and Delta variants, RBDdo: 1:1 mixture of SARS-COV-2 Spike RBD protein of delta and omicron variants. Numbers below the time axis indicate amount of injected pharmacologically active ingredient for prime/boost 1/boost 2. All RBD protein were adjuvanted with Alhydrogel (equivalent to 100 μg of Aluminum). Bar height represents the geometric mean of the individual values of each group. Prime boost regimen. Left panel of each FIGS. Sep. 9, 2020 and Mar. 3, 2020 μg dosing): RNA prime, RNA boost, Middle panel of each FIG. Sep. 15, 2020 μg and Mar. 5, 2020 μg dosing): RNA prime RBD boost regimen, Right panels of each FIGS. 15/15/20 and May 5, 2020 μg dosing): RBD prime, RBD boost. Dashed bars indicate pre-boost titers.

FIG. 8 shows KV-0620 Cell Line Selection Workflow.

FIG. 9 shows Western Blot Confirmation of KV-0620 Expression Levels 48 hours Post-Transfection.

FIG. 10 shows ELISA Scatter Diagram for Cell Pool Screening

FIG. 11 shows expression level of KV-0620. N means non-reducing condition; Lane 1: Precision Plus Protein™ Dual Color Standards (Bio-Rad, Cat.No. 161037.04); Lane 2: The supernatant of cell pool batch day 6 under non-reducing condition (Sample loading: 40 μl).

### DETAILED DESCRIPTION OF THE INVENTION

Booster immunizations can lead to rapid induction of protective immunity against pathogens (e.g. <7 days after the booster dose). This rapid response means that the re-focusing booster immunization can be administered about 1 week prior to an event that might require an activate immune status. For instance, a subject can be fully vaccinated (hetero- or homologous prime/boost regime) against the currently dominating SARS-COV-2 variant that does not affect the subjects now. However, for newly emerging SARS-COV-2 variants of concern the subject's immune response can be mobilized rapidly by a re-focusing boost immunization so that e.g., hospitalization in an immune-alert state is given. The re-focusing boost can further be administered if the immune status of subject is in question. The disclosed compositions and methods provide non-mRNA booster vaccines, particularly protein-based vaccines, which have characteristics different from those of mRNA vaccines, especially in terms of duration of immunity.

The initial animal efficacy studies described in Section 8 demonstrated that the protein formulations generate a robust antibody response targeting various SARS-COV-2 variants, including the B.1.617.2 (Delta) and B.1.1.529 (Omicron BA.1) variants, while toxicity markers indicate light and transient stimulation of inflammatory responses. If administered to mice in a heterologous RNA-prime/protein-booster regimen, the protein booster induced an order of magnitude enhancement of antibody titers compared to protein-only or RNA-only injected cohorts at any concentration tested. A prime/boost/break/re-boost study demonstrated the re-boosting of neutralizing antibody response following a decline from peak levels for any combination of initial immunization regimen tested (RNA/RNA, RNA/protein, protein/protein), inducing up to 65-fold antibody responses.

### A. DEFINITIONS

As used herein, the term “adjuvant” refers to a compound or mixture that enhances an immune response.

“Affinity tags” as used herein are peptide sequences appended to proteins so that they can be purified from a crude biological source using an affinity technique.

“Covalent linkage”, refers to a bond or organic moiety that covalently links molecules (e.g. fusion proteins) to a non-cellular surface.

The term “child” is meant to be a person or a mammal between 0 months and 18 years of age and “young child” refers to a child <5 yrs. Old.

As used herein, the term “effective amount” or “therapeutically effective amount” means a dosage sufficient to treat, inhibit, or alleviate one or more symptoms of a disease state being treated or to otherwise provide a desired pharmacologic effect. The precise dosage will vary according to a variety of factors such as subject-dependent variables (e.g., age, immune system health, etc.), the disease, and the age of the subject.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art. The term “immunogenic composition” or “composition” means that the composition can induce an immune response and is therefore antigenic. By “immune response” means any reaction by the immune system. These reactions include the alteration in the activity of an organism's immune system in response to an antigen and can involve, for example, antibody production, induction of cell-mediated immunity, complement activation, or development of immunological tolerance. As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides and proteins.

### II. COMPOSITIONS

Compositions for re-focusing an immune response to a pathogen include a fusion of antigenic portions of the pathogen, an adjuvant and optionally, a carrier. The disclosed composition aim to refocus the immune response of a subject against a pathogen by immunizing the subject with only the antigenic portion/immunogenic determinant of that pathogen, where the subject has been previously infected by the pathogen, or vaccinated against the pathogen. Exemplary pathogens include beta coronaviruses, preferably, SARS—Co-V-2, MERS-COV, etc. The disclosed compositions take advantage of antigen known to elicit neutralizing antibodies against the pathogen, and only includes the epitope of that antigen.

a. Fusion Protein/Peptide Component

In an embodiment, the pharmaceutically active ingredient of the re-focusing boost vaccines include one or several recombinant protein molecules truncated yet functional (meaning the overall antigenic tertiary structure is retained) version of disease-related protein or its protein domain, a smaller subunit or specific or modified epitope or combination of shorter epitopes derived from that initial prime antigen (protein/peptide) sequence.

In an embodiment, the protein vaccine includes a protein subunit, such as the receptor binding domain (RBD) of a virion surface protein and preferably, does not include full length spike protein.

In an embodiment, the antigen protein or its subunit (i.e., RBD) is engineered in such a way so as to be arrayed in 3D space in a regular, repeated fashion in order to promote B cell receptor engagement, clustering, and activation.

The booster formulations preferably include more than one RBD sequence, in tandem as a fusion protein, for example, as a dimer, trimer, tetramer or oligomer including multiple RBD units, which can be the same or different (i.e., hetero-tandem (=two different RBD's), multi genetic fusion (more than 2 RBDs) and hetero-multi genetic fusion RBD), which can be represented by the general formula:

RBD₁-L₁-RBD₂-L₂-RBDₙ, Formula I

where RBD₁ represents a first RBD sequence of a coronavirus, RBD₂ represents a second RBD sequence of a coronavirus, n is an integer represented the number of a subsequent RBD sequence(s) and L₁ and L₂ are optional first and second linkers, respectively.

In some forms, the RBD₁ is from a first coronavirus and RBD₂ is from the same coronavirus and RBDₙ are provided in tandem, from a coronavirus which is different from the first coronavirus. This embodiment provides fusion peptides for boosting an immune response to more than one type of virus, for example, SARS—Co-V-2 and MERS-COV by presenting the antigenic peptides therefrom in hetero-tandem format as described herein.

In some embodiments, the coronavirus is a variant of SARS-COV-2, such as SARS-COV-2 B.1.1.7 (Alpha variant), SARS-COV-2 B.1.351 (Beta variant), SARS-COV-2 P.1 (Gamma variant), SARS-COV-2 B.1.617, SARS-COV-2 B.1.617.1 (Kappa variant), SARS-COV-2 B.1.621 (Mu variant), SARS-COV-2 B.1.617.2 (Delta variant), SARS-COV-2 B.1.617.3, and SARS-COV-2 B.1.1.529 (Omicron variant).

In some forms, the more than one RDB sequences in the dimer include at least two different RDB sequences (hetero-tandem) for example, RBD sequence from two different variants of the same virus, i.e., RBD₁ is not the same sequence as RBD₂. although they are both from the same type of virus, for example, SARS—Co-V-2.

An example for a hetero-tandem RBD sequence is SEQ ID NO:16. The booster formulations disclosed herein preferably include alum or a derivative thereof, including derivatives of SEQ ID NO:16 with conservative amino acid substitutions, as the adjuvant. In an embodiment, the antigen protein or its subunit (i.e., RBD) is arrayed by tandem genetic fusion to create a repeated RBD domain construct expressed as a single polypeptide chain. Examples include, but are not limited to, hetero-tandem (=two different RBD's), multi genetic fusion (more than 2 RBDs) and hetero multi genetic fusion RBD. An example for a hetero-tandem is demonstrated in the Examples.

Exemplary RDB peptides that can be included
in the disclosed formulations include:
(i) SEQ ID NO: 2;
(2)
(SEQ ID NO: 18)
EAKPSGSVVEQAEGVECDFSPLLSGTPPQVYNFKRLVFTNCNYNL
TKLLSLFSVNDFTCSQISPAAIASNCYSSLILDYFSYPLSMKSDL
SVSSAGPISQFNYKQSFSNPTCLILATVPHNLTTITKPLKYSYIN
KCSRLLSDDRTEVPQLVNANQYSPCVSIVPSTVWEDGDYYRKQLS
PLEGGGWLVASGSTVAMTEQLQMGFGITVQYGTDTNSVCPKLEFA
NDTKIASQLGN,
i.e., SEQ ID NO: 2 without the
secretion tag;
(3) SEQ ID NO: 4,
(4)
(SEQ ID NO: 19)
EGVECDFSPLLSGTPPQVYNFKRLVFTNCNYNLTKLLSLFSVNDF
TCSQISPAAIASNCYSSLILDYFSYPLSMKSDLSVSSAGPISQFN
YKQSFSNPTCLILATVPHNLTTITKPLKYSYINKCSRLLSDDRTE
VPQLVNANQYSPCVSIVPSTVWEDGDYYRKQLSPLEGGGWLVASG
STVAMTEQLQMGFGITVQYGTDTNSVCPKL
i.e., SEQ ID NO: 4 without the secretion tag;
(5)
(6) SEQ ID NO: 6,
(7)
(SEQ ID NO: 20)
EAKPSGSVVEQAEGVECDFSPLLSGTPPQVYNFKRLVFTNCNYNL
TKLLSLFSVNDFTCSQISPAAIASNCYSSLILDYFSYPLSMKSDL
SVSSAGPISQFNYKQSFSNPTCLILATVPHNLTTITKPLKYSYIN
KCSRLLSDDRTEVPQLVNANQYSPCVSIVPSTVWEDGDYYRKQLS
PLEGGGWLVASGSTVAMTEQLQMGFGITVQYGTDTNSVCPKLEFA
NDTKIASQLGNEAKPSGSVVEQAEGVECDFSPLLSGTPPQVYNFK
RLVFTNCNYNLTKLLSLFSVNDFTCSQISPAAIASNCYSSLILDY
FSYPLSMKSDLSVSSAGPISQFNYKQSFSNPTCLILATVPHNLTT
ITKPLKYSYINKCSRLLSDDRTEVPQLVNANQYSPCVSIVPSTVW
EDGDYYRKQLSPLEGGGWLVASGSTVAMTEQLQMGFGITVQYGTD
TNSVCPKLEFANDTKIASQLGN,
i.e., SEQ ID NO: 6 without the secretion tag;
(8) SEQ ID NO: 8;
(9)
(SEQ ID NO: 21)
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGENCYFPLQSYGFQ
PTNGVGYQPYRVVVLSFELLHAPATVCGPKKSTNLVKNKAVNFNF
NGL,
i.e., SEQ ID NO: 8 without the secretion tag;
(10) SEQ ID NO: 10;
(11)
(SEQ ID NO: 22)
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGNIADYNYKLPDDFTGCVIAWNSNNLDSKVGGNYNYLY
RLFRKSNLKPFERDISTEIYQAGSTPCNGVKGFNCYFPLQSYGFQ
PTYGVGYQPYRVVVLSFELLHAPATVCGPKKSTNLVKNKAVNFNF
NGL,
i.e., SEQ ID NO: 10 without the secretion tag;
(12) SEQ ID NO: 12;
(13)
(SEQ ID NO: 23)
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNLVKNKAVNFNF
NGL,
i.e., SEQ ID NO: 12 without the secretion tag;
(14) SEQ ID NO: 15;
or
(15) SEQ ID NO: 16,

or polypeptide variants of SEQ ID Nos: 2, 4, 6, 8, 12, 15, 16, or a functional variant thereof having more than 90%, 91%, 92%, 93%, 94%, 95%, 96%, 97%, 98%, or 99% identical to the amino acid sequence of SEQ ID Nos: 2, 4, 6, 8, 10, 12, 15 or 16 or 18-23.

One preferred embodiment provides a composition containing an adjuvant such as alum, and SEQ ID NO:16 (herein KV-0620) or a functional variant thereof having more than 90%, 91%, 92%, 93%, 94%, 95%, 96%, 97%, 98%, or 99% identical to the amino acid sequence of SEQ ID NO; 16 in an effective amount to increase neutralizing antibodies in subject previously primed with non-protein/peptide vaccine (such as mRNA or adenovirus delivered antigen) against SARS-COV-2. KV-0620, which is a heterologous fusion dimeric antigen, including the RBD (residues R319-K537) of SARS-COV-2 Delta (B.1.617.2) and SARS-COV-2 Omicron (B.1.1.529, BA.1).

The optional L₁ and any subsequent linkers used to separate RBD moeities in the fusion protein/peptide are preferably peptide linkers sequences which are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Exemplary flexible peptides/polypeptides include, but are not limited to, the amino acid sequences Gly-Ser, Gly-Ser-Gly-Ser (SEQ ID NO:28), Ala-Ser, Gly-Gly-Gly-Ser (SEQ ID NO:29), (Gly4-Ser) 3 (SEQ ID NO:22), and (Gly4-Ser) 4 (SEQ ID NO:23), GSGSGSGS (SEQ ID NO: 24) and SGSG (SEQ ID NO:25). Additional flexible peptide/polypeptide sequences are well known in the art. In some forms, L₁ is flexible peptide modified to include a cysteine residue at its N- or C-terminus, for example, CGGSGSGSG (SEQ ID NO:26) or GSGC (SEQ ID NO:27).

### B. Carrier Materials

The antigenic protein or peptide is preferably presented on carrier. Suitable carriers include, but are not limited to anionic liposome, dendrimer, polynucleotide, synthetic nanoparticle, modified dendrimer nanoparticle, microgel, hydrogel, etc. The carrier may also be an adjuvant like Alum derivatives (AlHydrogel or AdjuPhos).

In an embodiment, the antigen protein or its subunit (i.e., RBD) is used without conjugation/covalent linkage to a carrier moiety.

### C. Adjuvants

The disclosed compositions include one or more adjuvants. Adjuvants are known.

Exemplary adjuvants include, but are not limited to, aluminum hydroxide (alum), aluminum phosphate, emulsion adjuvants, MF59, and AS03. LR agonists have been extensively studied as vaccine adjuvants. CpG, Poly I: C, glucopyranosyl lipid A (GLA), and resiquimod (R848) are agonists for TLR9, TLR3, TLR4, and TLR7/8, respectively. Exemplary CpG adjuvants that may be used in the disclosed compositions include, but at not limited to, CpG 1018 and CpG 1018 on Alum. In one preferred embodiment, the adjuvant is an Alum or alum derivative type adjuvant, such an aluminum hydroxide/oxyghydride gel (AIHYDROGEL® (aluminum hydroxide wet gel suspension) or aluminium phosphate gel (Adju-Phos® (aluminum phosphate wet gel suspension. Croda International PLC))), which preferably should be the carrier, or in the carrier. Alhydrogel® is a semi-crystalline form of aluminium oxyhydroxide (AH). Adju-Phos® is an amorphous salt of aluminium hydroxyphosphate (AP) which has been specifically developed for use as an adjuvant in vaccines. The gel is a suspension of hydrated amorphous aluminium hydroxyphosphate nano/micron size crystal in loose aggregates. Shardlow, et al., Allergy Asthma Clin Immunol 14, 80 (2018). https://doi.org/10.1186/s13223-018-0305-2.

Oil-Emulsion Adjuvants include squalene-water emulsions, such as MF59 (5% Squalene, 0.5% Tween 80, and 0.5% Span 85, formulated into submicron particles using a microfluidizer). See, e.g., WO90/14837. and, Podda, Vaccine 19:2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s-n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below). Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also comprise a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739. Optionally, the ISCOMS can be devoid of additional detergent. See WO00/07621. A description of the development of saponin based adjuvants can be found at Barr, et al., “ISCOMs and other saponin based adjuvants”, Advanced Drug Delivery Reviews 32:247-27, 1998. See also Sjolander, et al., “Uptake and adjuvant activity of orally delivered saponin and ISCOM vaccines”, Advanced Drug Delivery Reviews 32:321-338, 1998.

Bioadhesives and mucoadhesives can also be used as adjuvants. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some aspects, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19:109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s--n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27:571-577, 2002 and Jones, “Resiquimod 3M”, Curr Opin Investig Drugs 4:214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

### III. METHODS OF MAKING AND USING

### A. Methods of Making

Methods of making fusion proteins/peptide are known in the art, and include, for example, chemical synthesis, and more preferably, recombinant production in a host cell.

To recombinantly produce a fusion peptide of Formula I, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a fusion proteins of Formula I. Regulatory sequences (also referred to herein as expression control sequences) typically do not encode a gene product, but instead affect the expression of the nucleic acid sequences to which they are operably linked. The nucleotide sequences encoding the fusion protein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector is preferably an expression vector in which the DNA sequence encoding the fusion protein is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the fusion protein. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Examples of suitable promoters for directing the transcription of the DNA in mammalian cells are the SV40 promoter (Subramani et al., Mol. Cell. Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins/peptides of Formula I. Viral based expression systems are well known in the art and include, but are not limited to, baculoviral, SV40, retroviral, or vaccinia based viral vectors.

The expressed tagged or fusion proteins produced by the cells may be recovered from the culture medium by conventional procedures including separating the host cells from the medium by centrifugation or filtration, releasing the fusion protein by mechanical cell disruption, such as ultrasonication or pressure, precipitating the protein aqueous components of the supernatant or filtrate by means of a salt, e.g. ammonium sulphate. After sonication a suitable concentration of NaCl can be added to further decrease the ability of host cell contaminants to bind to the cation exchange matrix. After cation-exchange chromatography the fusion protein may be eluted in a salt gradient and eluate fractions containing the fusion protein are collected. In some preferred forms, fusion protein is captured from lysate through its His tag. So IMAC (immobilized metal affinity chromatography) was used and then, after concentration of protein-containing fractions, they are subjected to size exclusion chromatography (SEC) for final purification. In particularly preferred embodiments for nanobody purification, the nanobody is purified from the periplasmic space, where the host cell is bacteria, for example, E. coli. This would include (1) centrifugation, (2) osmotic shock to release the protein from the cell wall compartment, (3) IMAC (Immobilized Metal Ion Affinity Chromatography), (4) SEC (Size Exclusion Chromatography).

In some forms, fusion peptides including at least two RBD sequences in tandem, are arrayed or presented on a suitable carrier through electrostatic attraction including but not limited to the following: electrostatic immobilization of an antigen with a positive charge in the applied buffer or with a genetically fused tag coding for a highly basic peptide sequence on a negatively charged carrier (e.g. anionic liposome, dendrimer, polynucleotide or synthetic nanoparticle); and electrostatic immobilization of an antigen with a negative charge in the applied buffer or with a genetically fused tag coding for an acidic peptide sequence on a positively charged carrier (e.g. cationic liposome, dendrimer or synthetic nanoparticle, Alhydrogel and Adjuphos).

In some forms, the fusion peptide including at least two RBD sequences in tandem are arrayed or presented on a suitable carrier through a covalent linkage for example, through a reaction with a bi- or multifunctional cross-linker including, but not limited to glutaraldehyde, formaldehyde, CDI, and di- or oligo-NHS-esters.

In some forms, prior to its application the fusion peptide including at least two RBD sequences in tandem is physiosorbed on an adjuvant (i.e., alum).

In some forms, the carrier's surface on which the fusion peptide including at least two RBD sequences in tandem is arrayed or presented by one of the methods specified above is that of a synthetic nanoparticle, such as those produced by alkyl-modified dendrimer-based materials (modified dendrimer nanoparticle, MDNP), by self-assembly of a polynucleotides and alkyl-modified dendrimers, by self-assembly of a polynucleotides and cationic and neutral lipids, or by self-assembly of a polynucleotides, alkyl-modified dendrimers and appropriately charged lipids. Conjugation results in the linkage of the fusion peptide including at least two RBD sequences in tandem to one or several of the following moieties: an anionic, neutral or cationic lipid; a PEG moiety anchored to an anionic, neutral or cationic lipid; the backbone or functional group (e.g., amines) of the dendron.

In some forms, the carrier's surface on which the fusion peptide including at least two RBD sequences in tandem) is arrayed or presented by one of the methods specified above is that of a liposome consisting of anionic, neutral or cationic lipids or a mixture of these. Conjugation results in the linkage of the fusion peptide including at least two RBD sequences in tandem, to one or several of the following moieties: the polar head group or the aliphatic chain of a phospholipid; the core or the hydroxyl group of a sterol derived lipid; the polar head group of a saccharolipid, and the polar head group of a sphingolipid.

In some forms, the carrier on which the the fusion peptide including at least two RBD sequences in tandem is arrayed or presented by one of the methods specified above contains one or several moieties with adjuvanting or other immune-stimulating properties including, but not limited to: incorporation of an adjuvanting lipid like monophosphoryl lipid A and its derivatives, D-(+)-trehalose 6,6′-dibehenate, and cationic lipids like dimethyldioctadecylammonium into a liposome or a modified dendrimer nanoparticle; incorporation of a CpG-oligonucleotide or a RNA molecule in a liposome or a modified dendrimer nanoparticle; and conjugation of a CD4+T cell-activating helper peptide (e.g. PADRE sequence AKFVAAWTLKAAA (SEQ ID NO:13) to a self-assembling carrier protein, a liposome-forming lipid or a modified dendrimer nanoparticle.

In some forms, the fusion peptide including at least two RBD sequences in tandem is expressed as a genetic fusion product with an adjuvanting or otherwise immune-stimulating protein or peptide moiety, including but not limited to: a CD4+T cell-activating helper peptide (e.g., PADRE sequence AKFVAAWTLKAAA) (SEQ ID NO:13); and a protein with proven adjuvanting properties, e.g., keyhole limpet hemocyanin (KLH), and Concholepas concholepas hemocyanin (CCH).

In some forms, the fusion peptide including at least two RBD sequences in tandem is conjugated to one or several lipid anchor moieties prior to mixing with a in a liposome or a modified dendrimer nanoparticle.

In some forms, the fusion peptide including at least two RBD sequences in tandem is arrayed by conjugation to a microgel or hydrogel using the above-described conjugation methods.

In some forms, the fusion peptide including at least two RBD sequences in tandem is arrayed by conjugation to a DNA origami nanostructure.

In some forms, prior to its application formulation containing the fusion peptide including at least two RBD sequences in tandem is mixed with specific depot-forming adjuvants such as squalene/water and other nanoparticulate delivery systems.

### B. Methods of Using

The disclosed re-focusing boost formulations are used to augment an existing, yet not sufficiently protective immune response to effectively neutralize a pathogen of interest in a mammalian host organism. An exemplary embodiment is an adjuvanted RBD of coronaviruses (CoV), such as SARS-CoV-2, MERS-COV, or HCoVs vaccine for re-focusing boost immunization. The subject to whom the composition is administered is a subject previously infected with the virus or previously vaccinated against the virus using a vaccine (i.e., prime vaccine) in which the antigen is delivered using a virus such as adenovirus, RNA, such as mRNA, or a protein or peptide. In a preferred embodiment, the disclosed methods boost an immune response against a pathogen in a subject previously infected by the pathogen or vaccinated against the pathogen using a vaccine which provides antigen in a form delivered by an attenuated live virus, a vector delivered antigen for example antigen delivered via adenoviral delivery or nucleic acid delivery. In one embodiment, the subject has previously received a prime vaccine including a polynucleotide encoding a full-length antigenic protein or only minimally truncated disease-related protein or protein domain. The minimally truncated disease-related protein or protein domain may be a protein that retains the overall antigenic tertiary structure. The polynucleotide may be a molecule that encodes the full-sized form of a pathogen antigen. For example, the polynucleotide may be the complete coding sequence of a virion surface protein. The polynucleotide may encode the first antigenic protein or the second antigenic protein that comprises a viral Spike protein, or a fragment thereof. The viral Spike protein may be a peptide or polypeptide corresponding to one or more antigenic determinants of the receptor binding domain of the SARS-COV Spike protein, SARS-COV-2 Spike protein, or MERS-COV Spike protein. The receptor binding domain of the SARS-COV spike protein, SARS-COV-2 Spike protein, or MERS-COV Spike protein may be 10-20 amino acid residues in length, and may contain more than one peptide determinants of up to about 30-50 residues or more. A booster is preferably given when a person has completed their vaccine series, and protection against the virus has decreased over time.

The time between priming and boosting are typically known and publicly available. For example, the FDA has provided boosting intervals for SARS-CoV-2 immunization, ranging from 2 months (Jenssen (J &J) since completing primary vaccination, to at least 5 months (Moderna or Pfizer-BioNtech mRNA vaccines).

The disclosed compositions can be administered using any suitable administration route. Dosage forms for administration by parenteral (intramuscular (IM), intraperitoneal (IP), intravenous (IV) or subcutaneous injection (SC)), or transmucosal (nasal, vaginal, pulmonary, or rectal) routes of administration can be formulated. In the most preferred embodiments, the immunizing virus is delivered peripherally by intranasally or by intramuscular injection, and the booster formulation is delivered by local injection.

The disclosed formulations are administered to a subject in need thereof such as a human subject. The subject preferably had been primed, either by an infection with the pathogen or an immunization against the pathogen, either of which result in a balanced T cell and B cell immune response to the pathogen.

The subject can be about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger. In some embodiments, the subject is a young adult between the ages of about 20 years and about 50 years (e.g., about 20, 25, 30, 35, 40, 45 or 50 years old). In some embodiments, the subject is an elderly subject about 60 years old, about 70 years old, or older (e.g., about 60, 65, 70, 75, 80, 85 or 90 years old).

In one preferred embodiment, the subject previously was primed by immunization against the pathogen of interest, using a vaccine which delivers antigen via mRNA.

The disclosed refocusing booster formulations produce prophylactically- and/or therapeutically efficacious levels, concentrations and/or titers of antigen-specific, preferably, neutralizing antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). Additional methods for titer determinations include pseudo-virus neutralization assays and RBD-hACE2 blocking assays. In exemplary embodiments, antibody titer is determined or measured by neutralization assay, e.g., by microneutralization assay. In certain aspects, antibody titer measurement is expressed as a ratio, such as 1:40, 1:100, etc. In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of μg/ml or are measured in units of IU/L (International Units per liter) or mIU/ml (milli International Units per ml). In exemplary embodiments, an efficacious vaccine produces >0.05 μg/ml, >0.1 μg/ml, >0.2 μg/ml, >0.35 μg/ml, >0.5 μg/ml, >1 μg/ml, >2 μg/ml, >5 μg/ml or >10 μg/ml and up to >100 μg/mL. In exemplary embodiments, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

As proof-of-concept, (a) RBDs of several SARS-COV-2 strains (Wuhan, SARS-COV-2 B.1.351, SARS-COV-2 B.1.617.2), the RBD of MERS-COV and HCoV-HKU1 were produced in an XtenCHO expression system and (b) a protein subunit booster vaccine candidate (KV-0620) based on the receptor binding domain (RBD) of the SARS-COV-2 Spike(S) glycoprotein (residues R319-K537), which is responsible for mediating cell entry and interaction with host receptor angiotensin-converting enzyme 2 (ACE2). The Spike RBD antigens are produced using transient plasmid transfection of a serum-free suspension XtenCHO cell line sourced from CLS Cell Lines Service GmbH. Research grade material was produced using a research cell bank. Briefly, purified plasmid from the respective antigen expression cassettes is transfected into the XtenCHO cell line using FectoPro transfection reagent. The cells are monitored for cell viability, cell density and cultured under low-endotoxin conditions for up to 13 days (312 hours). The protein antigens are secreted into the culture media and affinity purified.

The protein RBD constructs of the SARS-COV-2 variants and the MERS-COV were formulated with aluminum hydroxide adjuvant. Briefly, 25 μg of RBD antigen in TBS were mixed with 750 μg of 2% Alhydrogel (InvivoGen) and incubated with shaking for 30 min at room-temperature to allow adsorption, followed by storage at 4° C. All vaccines were diluted in TBS before administration. C57BL/6 mice (n=5) were immunized with the indicated vaccines at 15, 3, or 0.6 μg dose levels, and serum collected 2 weeks later. Direct mouse IgG ELISA was performed on recombinant SARS-COV-2 A) Wuhan, B) MERS or C) B.1.351 RBD protein.

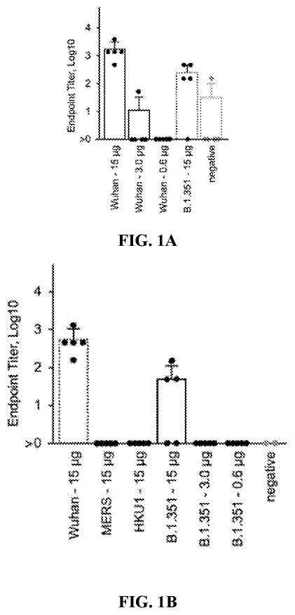

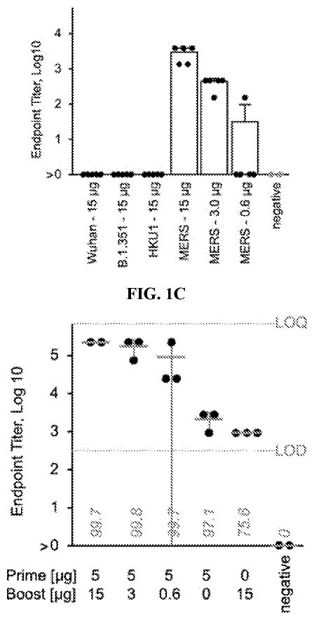

Serum samples from mice vaccinated with the SARS-COV-2 RBD exhibited positive antibody titers against the SARS-COV-2 RBDs (FIGS. 1A and 1C). The antibody titers were specific for the corresponding SARS-COV-2 RBD; immunity versus old (FIG. 1A) and newly emerging SARS-COV-2 strains was obtained (FIG. 1B). Lastly, a MERS-COV immunization could be achieved by high antibody titers against the MERS RBD (FIG. 1C). These results indicate that adjuvant formulated RBD proteins can induce broad, and specific immune responses with high antibody titers.

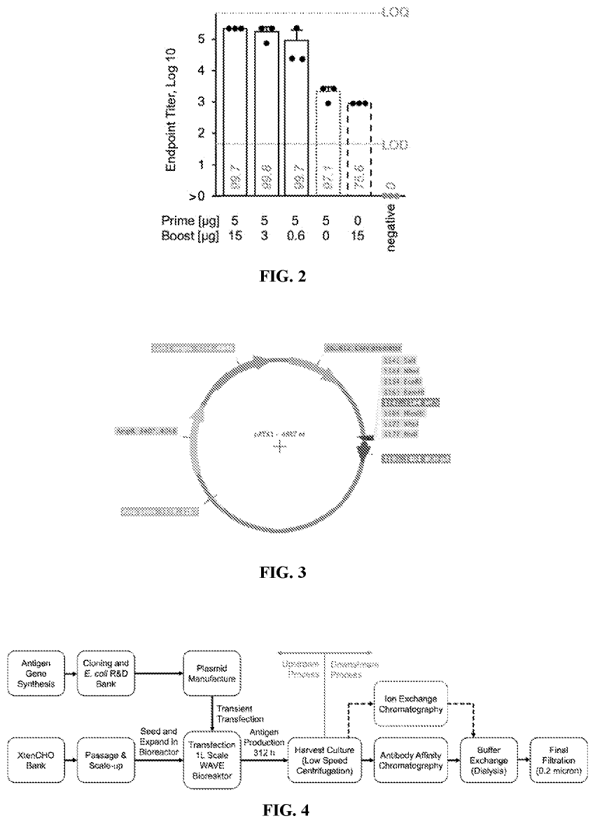

The RNA prime was expressed in vitro using BHK cells, purified by lithium chloride precipitation and confirmed by immunoblotting. Upon validation, RNA was formulated with a proprietary delivery molecule and injected into C57BL/6 mice at a 5 μg dose. The formulated adjuvanted RBD protein vaccine was injected into C57BL/6 mice at a 0.6-15 μg doses. Mice were primed with full-length B.1.351 Spike RNA vaccine and boosted with Wuhan RBD protein at the indicated dose levels 38 days later. Serum was collected 3 weeks after boost, and direct mouse IgG ELISA was performed on recombinant SARS-COV-2 Wuhan RBD protein. The grey values at the bottom inside the graph indicate surrogate VNT % inhibition. Serum samples from mice vaccinated with the SARS-COV-2 RBD exhibited positive antibody titers against both the SARS-COV-2 RBD (FIG. 2).

Current data show that within three weeks after the re-focusing boost immunization a nearly two orders of magnitude antibody level elevation could be detected along with low levels of inflammation response.

### IV. Kits

Kits are also disclosed. The kit can include a single dose or a plurality of doses of a composition including a fusion protein/peptide of Formula I and instructions for administering the compositions. Specifically, the instructions direct that an effective amount of the composition be administered to an individual at risk of exposure to one or more respiratory pathogens such as severe acute respiratory syndrome (SARS) virus. The composition can be formulated as described above with reference to a particular treatment method and can be packaged in any convenient manner.

The present invention will be further understood by reference to the following non-limiting examples.

Particularly preferred embodiments are exemplified below.

### Examples

### Materials and Methods

### Synthesis and Formulation of the RBD Protein Boost Vaccine Component

List of selected RBD Sequences
Expression of tag-free RBDs in XtenCHO cells
Nucleotide Sequence of MERS-CoV Spike RBD
Antigen; 256 AAs, MW = 27.98 kDa
(optimized for CHO)
(SEQ ID NO: 1)
gaattcgccgccaccATGTACAGGATGCAGCTGCTGTCCTGCATC
GCCCTGAGCCTGGCCCTGGTGACAAATTCCGAGGCCAAGCCTAGC
GGCAGCGTGGTGGAGCAGGCCGAGGGAGTGGAGTGCGACTTCTCC
CCCCTGCTGAGCGGCACCCCCCCACAAGTGTACAACTTCAAGAGA
CTGGTGTTCACAAACTGTAATTACAACCTGACCAAGCTGCTGTCC
CTGTTCTCCGTGAATGATTTCACCTGCAGCCAGATCTCCCCTGCC
GCCATCGCCTCCAACTGCTACTCCAGCCTGATCCTGGATTACTTC
TCCTACCCCCTGAGCATGAAGTCCGATCTGAGCGTGAGCTCCGCC
GGCCCCATCAGCCAGTTCAACTACAAGCAGTCCTTCTCCAACCCT
ACCTGTCTGATCCTGGCCACCGTGCCCCACAATCTGACCACCATC
ACCAAGCCCCTGAAGTACTCCTACATCAATAAGTGTAGCAGGCTG
CTGTCCGACGATAGAACAGAGGTGCCTCAGCTGGTGAATGCCAAC
CAGTACAGCCCCTGCGTGAGCATCGTGCCTAGCACCGTGTGGGAG
GATGGCGACTACTACAGGAAGCAGCTGTCCCCCCTGGAGGGCGGC
GGATGGCTGGTTGCTTCCGGCAGCACCGTGGCCATGACCGAGCAG
CTGCAGATGGGCTTCGGCATCACCGTGCAGTACGGCACAGACACC
AATAGCGTGTGTCCTAAGCTGGAGTTCGCCAACGATACAAAGATC
GCCAGCCAGCTGGGCAATtgagcggccgc.
Amino Acid Sequence of MERS-CoV Spike RBD
Antigen; 256 AAs, MW = 27.98 kDa; bolded and
italicized: secretion-tag
(SEQ ID NO: 2)
MYRMQLLSCIALSLALVTNSEAKPSGSVVEQAEGVECDFSPLLSG
TPPQVYNFKRLVFTNCNYNLTKLLSLFSVNDFTCSQISPAAIASN
CYSSLILDYFSYPLSMKSDLSVSSAGPISQFNYKQSFSNPTCLIL
ATVPHNLTTITKPLKYSYINKCSRLLSDDRTEVPQLVNANQYSPC
VSIVPSTVWEDGDYYRKQLSPLEGGGWLVASGSTVAMTEQLQMGF
GITVQYGTDTNSVCPKLEFANDTKIASQLGN.
Nucleotide Sequence of MERS-CoV Spike RBD 2
Antigen (optimized for CHO).
(SEQ ID NO: 3)
gaattcgccgccaccATGTACAGGATGCAGCTGCTGAGCTGTATC
GCCCTGAGCCTGGCCCTGGTGACCAATAGCCAGGCCGAGGGCGTG
GAGTGTGACTTTTCCCCTCTGCTGAGCGGCACCCCTCCTCAGGTG
TACAATTTCAAGAGACTGGTGTTCACAAACTGCAATTACAACCTG
ACAAAGCTGCTGAGCCTGTTCTCCGTGAATGACTTCACATGCAGC
CAGATCAGCCCCGCCGCCATCGCCAGCAACTGCTACTCCTCCCTG
ATCCTGGACTACTTCTCCTACCCTCTGTCCATGAAGAGCGATCTG
AGCGTGTCCAGCGCCGGCCCCATCTCCCAGTTTAACTACAAGCAG
TCCTTCAGCAATCCTACATGCCTGATCCTGGCCACAGTGCCCCAC
AATCTGACCACCATCACCAAGCCCCTGAAGTACAGCTACATCAAC
AAGTGCTCCAGACTGCTGAGCGACGATAGGACCGAGGTGCCTCAG
CTGGTGAATGCCAATCAGTACTCCCCTTGTGTGTCCATCGTGCCT
TCCACAGTGTGGGAGGACGGCGACTACTACAGGAAGCAGCTGTCC
CCTCTGGAGGGCGGCGGCTGGCTGGTTGCTAGCGGATCCACCGTG
GCCATGACCGAGCAGCTGCAGATGGGCTTCGGCATCACAGTGCAG
TACGGCACAGACACCAACAGCGTGTGTCCCAAGCTGtgageggcc
gc.
Amino Acid of MERS-CoV Spike RBD 2 Antigen;
232 AAs, MW = 25.51 kDa; bolded and
italicized: secretion-tag
(SEQ ID NO: 4)
MYRMQLLSCIALSLALVTNSQAEGVECDFSPLLSGTPPQVYNFKR
LVFTNCNYNLTKLLSLFSVNDFTCSQISPAAIASNCYSSLILDYF
SYPLSMKSDLSVSSAGPISQFNYKQSFSNPTCLILATVPHNLTTI
TKPLKYSYINKCSRLLSDDRTEVPQLVNANQYSPCVSIVPSTVWE
DGDYYRKQLSPLEGGGWLVASGSTVAMTEQLQMGFGITVQYGTDT
NSVCPKL.
Nucleotide Sequence of MERS-CoV Spike RBD
Dimer Antigen (optimized for CHO)
(SEQ ID NO: 5)
gaattcgccgccaccATGTACAGGATGCAGCTGCTGTCCTGCATC
GCCCTGAGCCTGGCCCTGGTGACAAATTCCGAGGCCAAGCCTAGC
GGCAGCGTGGTGGAGCAGGCCGAGGGAGTGGAGTGCGACTTCTCC
CCCCTGCTGAGCGGCACCCCCCCACAAGTGTACAACTTCAAGAGA
CTGGTGTTCACAAACTGTAATTACAACCTGACCAAGCTGCTGTCC
CTGTTCTCCGTGAATGATTTCACCTGCAGCCAGATCTCCCCTGCC
GCCATCGCCTCCAACTGCTACTCCAGCCTGATCCTGGATTACTTC
TCCTACCCCCTGAGCATGAAGTCCGATCTGAGCGTGAGCTCCGCC
GGCCCCATCAGCCAGTTCAACTACAAGCAGTCCTTCTCCAACCCT
ACCTGTCTGATCCTGGCCACCGTGCCCCACAATCTGACCACCATC
ACCAAGCCCCTGAAGTACTCCTACATCAATAAGTGTAGCAGGCTG
CTGTCCGACGATAGAACAGAGGTGCCTCAGCTGGTGAATGCCAAC
CAGTACAGCCCCTGCGTGAGCATCGTGCCTAGCACCGTGTGGGAG
GATGGCGACTACTACAGGAAGCAGCTGTCCCCCCTGGAGGGCGGC
GGATGGCTGGTTGCTTCCGGCAGCACCGTGGCCATGACCGAGCAG
CTGCAGATGGGCTTCGGCATCACCGTGCAGTACGGCACAGACACC
AATAGCGTGTGTCCTAAGCTGGAGTTCGCCAACGATACAAAGATC
GCCAGCCAGCTGGGCAATGAGGCCAAGCCCTCCGGCTCCGTGGTG
GAGCAAGCCGAGGGCGTGGAGTGTGACTTCTCCCCTCTGCTGAGC
GGAACCCCTCCTCAGGTGTACAACTTTAAGAGACTGGTCTTCACC
AACTGCAACTACAATCTGACAAAGCTGCTGAGCCTGTTCAGCGTG
AACGACTTCACCTGTAGCCAGATCAGCCCCGCCGCCATCGCTAGC
AACTGTTACTCCTCCCTGATCCTGGACTACTTTTCCTACCCCCTC
TCCATGAAGTCCGACCTGAGCGTGTCCTCCGCCGGCCCAATCAGC
CAGTTTAACTACAAGCAAAGCTTTAGCAACCCTACCTGCCTGATC
CTGGCTACAGTGCCTCACAATCTGACAACAATCACAAAGCCTCTG
AAGTACAGCTACATCAACAAGTGCAGCAGACTGCTGTCCGATGAC
AGGACCGAGGTGCCCCAGCTGGTGAACGCCAATCAGTACAGCCCA
TGCGTGAGCATTGTGCCTAGCACAGTGTGGGAGGACGGCGATTAC
TACAGAAAGCAGCTGAGCCCCCTGGAGGGAGGCGGCTGGCTGGTT
GCATCCGGCTCCACCGTGGCCATGACAGAGCAGCTGCAAATGGGC
TTCGGAATCACCGTGCAATACGGCACAGATACAAACTCCGTGTGC
CCCAAGCTGGAGTTTGCCAACGATACCAAGATCGCCTCCCAGCTG
GGCAACtgagcggccgc.
Amino Acid Sequence of MERS-CoV Spike RBD
Antigen; 492 AAs, MW = 53.74 kDa; bolded and
italicized: secretion-tag:
(SEQ ID NO: 6)
MYRMQLLSCIALSLALVTNSEAKPSGSVVEQAEGVECDFSPLLSG
TPPQVYNFKRLVFTNCNYNLTKLLSLFSVNDFTCSQISPAAIASN
CYSSLILDYFSYPLSMKSDLSVSSAGPISQFNYKQSFSNPTCLIL
ATVPHNLTTITKPLKYSYINKCSRLLSDDRTEVPQLVNANQYSPC
VSIVPSTVWEDGDYYRKQLSPLEGGGWLVASGSTVAMTEQLQMGF
GITVQYGTDTNSVCPKLEFANDTKIASQLGNEAKPSGSVVEQAEG
VECDFSPLLSGTPPQVYNFKRLVFTNCNYNLTKLLSLFSVNDFTC
SQISPAAIASNCYSSLILDYFSYPLSMKSDLSVSSAGPISQFNYK
QSFSNPTCLILATVPHNLTTITKPLKYSYINKCSRLLSDDRTEVP
QLVNANQYSPCVSIVPSTVWEDGDYYRKQLSPLEGGGWLVASGST
VAMTEQLQMGFGITVQYGTDTNSVCPKLEFANDTKIASQLGN.
Nucleotide Sequence of SARS-CoV-2 Wuhan
Spike RBD Antigen; 244 AAs, MW = 27.29 kDa
(optimized for CHO)
(SEQ ID NO: 7)
gaattcgccgccaccATGCCTCTGCTGCTGCTGCTCCCCCTGCTG
TGGGCCGGAGCTCTGGCTAGGGTGCAGCCCACCGAGAGCATCGTG
AGGTTCCCCAATATCACAAATCTGTGTCCCTTCGGCGAGGTGTTT
AACGCCACCAGGTTTGCCTCCGTGTACGCCTGGAATAGGAAGAGA
ATCAGCAATTGTGTGGCCGACTACAGCGTGCTGTACAATTCCGCC
AGCTTCTCCACCTTCAAGTGCTACGGCGTGAGCCCCACCAAGCTG
AATGACCTGTGTTTTACCAATGTGTACGCCGACAGCTTCGTGATC
AGGGGCGATGAGGTGAGGCAGATCGCCCCCGGCCAGACAGGCAAG
ATCGCCGATTACAATTACAAGCTGCCTGATGATTTTACCGGCTGT
GTGATCGCCTGGAATAGCAATAACCTGGATAGCAAGGTGGGCGGC
AACTACAATTACCTGTACAGACTGTTTAGAAAGTCCAACCTGAAG
CCCTTCGAGAGGGACATCAGCACCGAGATCTACCAGGCCGGCTCC
ACACCTTGTAACGGCGTGGAGGGCTTCAACTGCTACTTTCCCCTG
CAGAGCTACGGCTTCCAGCCCACCAATGGCGTGGGCTACCAGCCT
TACAGAGTGGTGGTGCTGAGCTTTGAGCTGCTGCACGCCCCCGCC
ACCGTGTGTGGACCTAAGAAGAGCACCAATCTGGTGAAGAATAAG
GCCGTGAACTTTAACTTTAATGGCCTGtgaaagctt.
Amino Acid Sequence of SARS-CoV-2 Wuhan Spike
RBD Antigen; 244 AAs, MW = 27.29 kDa; bolded
and italicized = secretion-tag
(SEQ ID NO: 8)
MPLLLLLPLLWAGALARVQPTESIVRFPNITNLCPFGEVFNATRF
ASVYAWNRKRISNCVADYSVLYNSASFSTFKCYGVSPTKLNDLCF
TNVYADSFVIRGDEVRQIAPGQTGKIADYNYKLPDDFTGCVIAWN
SNNLDSKVGGNYNYLYRLFRKSNLKPFERDISTEIYQAGSTPCNG
VEGENCYFPLQSYGFQPTNGVGYQPYRVVVLSFELLHAPATVCGP
KKSTNLVKNKAVNFNFNGL.
Nucleotide Sequence of SARS-CoV-2 Beta
(B.1.351) Variant Spike
RBD Antigen (optimized for CHO)
(SEQ ID NO: 9)
gaattcgccgccaccATGCCTCTGCTGCTGCTGCTCCCCCTGCTG
TGGGCCGGAGCTCTGGCTAGGGTGCAGCCTACAGAGTCCATCGTG
AGGTTTCCTAACATCACAAACCTGTGTCCTTTTGGCGAGGTGTTT
AATGCCACAAGATTTGCCAGCGTGTACGCCTGGAATAGGAAGAGG
ATCAGCAATTGCGTGGCCGACTACTCCGTGCTGTACAATAGCGCC
AGCTTTTCCACCTTTAAGTGCTACGGCGTGAGCCCCACAAAGCTG
AATGACCTGTGTTTTACCAACGTGTACGCCGACAGCTTTGTGATC
AGGGGCGACGAGGTGAGACAGATCGCCCCCGGCCAGACCGGCAAT
ATCGCCGATTACAACTACAAGCTGCCTGACGATTTCACAGGCTGC
GTGATCGCCTGGAATAGCAACAATCTGGACAGCAAGGTGGGCGGC
AACTACAATTACCTGTACAGGCTGTTCAGAAAGTCCAACCTGAAG
CCCTTTGAGAGGGACATCTCCACAGAGATCTACCAGGCCGGCTCC
ACCCCCTGTAATGGCGTGAAGGGCTTTAACTGTTACTTTCCCCTG
CAGAGCTACGGCTTCCAGCCCACCTACGGCGTGGGCTACCAGCCC
TACAGAGTGGTGGTGCTGAGCTTCGAGCTGCTGCACGCCCCTGCC
ACCGTGTGCGGACCTAAGAAGAGCACCAACCTGGTGAAGAACAAG
GCCGTGAACTTTAACTTTAATGGCCTGtgaaagctt.
Amino acid Sequence of SARS-CoV-2 Beta (B.1.351)
Variant Spike RBD Antigen; 244 AAs,
MW = 27.33 kDa bolded and italicized:
secretion-tag
(SEQ ID NO: 10)
MPLLLLLPLLWAGALARVQPTESIVRFPNITNLCPFGEVENATRF
ASVYAWNRKRISNCVADYSVLYNSASFSTFKCYGVSPTKLNDLCF
TNVYADSFVIRGDEVRQIAPGQTGNIADYNYKLPDDFTGCVIAWN
SNNLDSKVGGNYNYLYRLFRKSNLKPFERDISTEIYQAGSTPCNG
VKGFNCYFPLQSYGFQPTYGVGYQPYRVVVLSFELLHAPATVCGP
KKSTNLVKNKAVNFNFNGL.
Nucleotide Sequence of humanized SARS-CoV-2
Delta (B.1.617.2) Variant Spike RBD Antigen
(optimized for CHO)
(SEQ ID NO: 11)
gaattcgccgccaccATGCCTCTGCTGCTGCTGCTCCCCCTGCTG
TGGGCCGGAGCTCTGGCTAGGGTGCAGCCCACCGAGAGCATCGTG
AGGTTCCCCAATATCACAAATCTGTGTCCCTTCGGCGAGGTGTTT
AACGCCACCAGGTTTGCCTCCGTGTACGCCTGGAATAGGAAGAGA
ATCAGCAATTGTGTGGCCGACTACAGCGTGCTGTACAATTCCGCC
AGCTTCTCCACCTTCAAGTGCTACGGCGTGAGCCCCACCAAGCTG
AATGACCTGTGTTTTACCAATGTGTACGCCGACAGCTTCGTGATC
AGGGGCGATGAGGTGAGGCAGATCGCCCCCGGCCAGACAGGCAAG
ATCGCCGATTACAATTACAAGCTGCCTGATGATTTTACCGGCTGT
GTGATCGCCTGGAATAGCAATAACCTGGATAGCAAGGTGGGCGGC
AACTACAATTACAGATACAGACTGTTTAGAAAGTCCAACCTGAAG
CCCTTCGAGAGGGACATCAGCACCGAGATCTACCAGGCCGGCTCC
AAGCCTTGTAACGGCGTGGAGGGCTTCAACTGCTACTTTCCCCTG
CAGAGCTACGGCTTCCAGCCCACCAATGGCGTGGGCTACCAGCCT
TACAGAGTGGTGGTGCTGAGCTTTGAGCTGCTGCACGCCCCCGCC
ACCGTGTGTGGACCTAAGAAGAGCACCAATCTGGTGAAGAATAAG
GCCGTGAACTTTAACTTTAATGGCCTGtgaaagctt.
Amino acid Sequence of SARS-CoV-2 Delta
(B.1.617.2) Variant Spike RBD Antigen;
244 AAs, MW = 27.36 kDa;
bolded and italicized: secretion-tag
(SEQ ID NO: 12)
MPLLLLLPLLWAGALARVQPTESIVRFPNITNLCPFGEVENATRF
ASVYAWNRKRISNCVADYSVLYNSASFSTFKCYGVSPTKLNDLCF
TNVYADSFVIRGDEVRQIAPGQTGKIADYNYKLPDDFTGCVIAWN
SNNLDSKVGGNYNYRYRLFRKSNLKPFERDISTEIYQAGSKPCNG
VEGENCYFPLQSYGFQPTNGVGYQPYRVVVLSFELLHAPATVCGP
KKSTNLVKNKAVNFNFNGL.
SARS-CoV-2 Omicron Variant (B.1.1.529,BA.1)
Spike RBD Antigen Nucleotide Sequence
(SEQ ID NO: 18)
GAATTCGCCGCCACCATGCCCCTGCTGCTGCTGCTCCCTCTGCTG
TGGGCCGGCGCTCTGGCTAGAGTGCAGCCTACAGAGAGCATCGTG
AGGTTCCCTAATATCACAAACCTGTGCCCTTTTGACGAGGTGTTC
AACGCCACAAGGTTTGCCTCCGTGTACGCCTGGAACAGAAAGAGA
ATCAGCAATTGTGTGGCCGATTACAGCGTGCTGTACAATCTGGCC
CCCTTTTTCACATTTAAGTGTTACGGCGTGTCCCCCACCAAGCTG
AATGATCTGTGCTTCACCAACGTGTACGCCGACAGCTTTGTGATC
AGAGGCGACGAGGTGAGACAGATCGCCCCTGGCCAGACCGGCAAC
ATCGCCGATTACAACTACAAGCTGCCCGATGACTTTACCGGCTGC
GTGATCGCCTGGAACTCCAACAAGCTGGACAGCAAGGTGTCCGGC
AACTACAACTACCTGTACAGGCTGTTCAGGAAGTCCAATCTGAAG
CCTTTCGAGAGAGATATCTCCACAGAGATCTACCAGGCCGGCAAC
AAGCCCTGCAATGGCGTGGCCGGCTTTAATTGTTACTTTCCTCTG
CGAAGCTACTCCTTTAGACCTACCTACGGCGTGGGCCACCAGCCT
TACAGAGTGGTGGTGCTGTCCTTTGAGCTGCTGCACGCCCCTGCC
ACAGTGTGTGGCCCCAAGAAGTCCACCAACCTGGTGAAGAACAAG
TGAGCGGCCGC.
SARS-CoV-2 Omicron Variant (B.1.1.529,BA.1)
Spike RBD Antigen Amino Acid Sequence Red
Secretion Tag bolded and italicized.
(SEQ ID NO: 19)
MPLLLLLPLLWAGALARVQPTESIVRFPNITNLCPFDEVFNATRF
ASVYAWNRKRISNCVADYSVLYNLAPFFTFKCYGVSPTKLNDLCF
TNVYADSFVIRGDEVRQIAPGQTGNIADYNYKLPDDFTGCVIAWN
SNKLDSKVSGNYNYLYRLFRKSNLKPFERDISTEIYQAGNKPCNG
VAGENCYFPLRSYSFRPTYGVGHQPYRVVVLSFELLHAPATVCGP
KKSTNLVKNK.

### RBD Protein Expression and Purification

XtenCHO Cell line. The CHO-K1 cell line used for development of XtenCHO cell line was obtained from CLS Cell Lines Service GmbH. The research and development was conducted with a research Mycoplasma free cell bank, and production with serum free (FBS) medium.

Expression of tag-free RBDs in XtoCHO cells.

The research materials produced to date wereprocured from ProteoGenix (ProteoGenix, 15 rue de la Haye, 67300, Schiltigheim, France). This included the gene synthesis and preparation of expression plasmids followed by recombinant antigen production and purification. The Spike RBD protein sequences were codon optimized for expression in Chinese hamster ovary cells (XtenCHO) and synthesized by artificial gene synthesis.

Each respective sequence was cloned into plasmid pTAX1 (FIG. 3) for expression in mammalian cells. Each antigen encoding plasmid was transfected into Top10 Each antigen encoding plasmid was transfected into One Shot™ TOP10 chemically competent E. coli purified plasmid qualified by confirmation of the Spike RBD gene based on restriction enzymatic digestion band patterns and DNA sequence analysis of the inserted gene region and tested for residual endotoxin levels.

Each coronavirus Spike RBD antigen is produced using transient plasmid transfection of a serum-free suspension XtenCHO cell line. Briefly, purified plasmid from the respective antigen expression cassettes is transfected into the XtenCHO cell line using FectoPro transfection reagent. The cells are monitored for cell viability, cell density and cultured for up to 13 days (312 hours). The Spike RBD antigens are secreted into the culture media. An overview of the upstream process is provided in FIG. 4.

The XtenCHO cells expression in combination with the IEX approach is scalable and applicable to GMP production phase.

The Spike RBD antigens are purified through ion exchange chromatography (IEX) or affinity chromatography using anti-RBD antibodies developed by ProteoGenix. The research grade antigen is then buffer exchanged into the formulation buffer. Additional process steps such as ion exchange chromatography can be implemented to enhance product purify.

### RBD-Protein Formulation

Proteins supplied by Proteogenix in PBS were adjuvanted prior to injection for all animal studies. Alhydrogel 2% stock solution (10 mg/ml) from InvivoGen was mixed 1:1 with protein at 0.3 μg/μL, followed by a 30 minute incubation period on ice. Sample diluted in PBS to appropriate concentration for injection.

### Animal Studies

### Mouse Experiments

Gene expression and immunogenicity analyses. All procedures were approved by Tiba's Institutional Animal Care and Use Committee. 4-8 week old female BALB/c mice were injected by bilateral i.m. injection in the leg muscles and sampled for SEAP expression approximately 16 h later by puncture of the submandibular vein for blood collection and subsequent serum isolation. Reporter gene expression was quantified by measurement of SEAP concentration using the Phospha-Light reporter gene assay system (Thermo Fisher Scientific, Waltham, MA). T cell responses were measured 11 days post-injection by removal of the spleen from necropsied animals followed by splenocyte isolation and restimulation with SARS-COV-2 derived peptides following methods essentially as described previously¹⁵, except that splenocytes were cultured directly on anti-IFN-gamma coated flat-bottom Nunc MaxiSorp plates (Thermo Fisher Scientific, Waltham, MA). IFN-gamma production by restimulated cells was measured by sandwich ELISA after decanting the cultured cells. Anti-spike titers were measured by endpoint dilution of serum on recombinant Spike-coated Nunc MaxiSorp plates (Thermo Fisher Scientific, Waltham, MA) using standard methods, with endpoint cutoffs determined by negative control serum from unimmunized animals.

### Synthesis and Formulation of the Protein Boost Vaccine Component

The phylogenetic analysis of coronavirus genomes has revealed that SARS-COV-2 is a member of the Betacoronavirus genus, which includes SARS-CoV, MERS-COV, as well as others identified in humans and diverse animal species. Generally, all coronaviruses use the homotrimeric spike glycoprotein (comprising a S1 subunit and S2 subunit in each spike monomer) located on the envelope to bind to their corresponding cellular receptor. Such binding triggers a cascade leading to the fusion between cell and viral membrane allowing the virus the cell entry. Consequently, the receptor-binding domain (RBD) of CoV spike(S) proteins can serve as a valuable target for developing antibodies, entry inhibitors and vaccines.

### Selected RBD Sequences

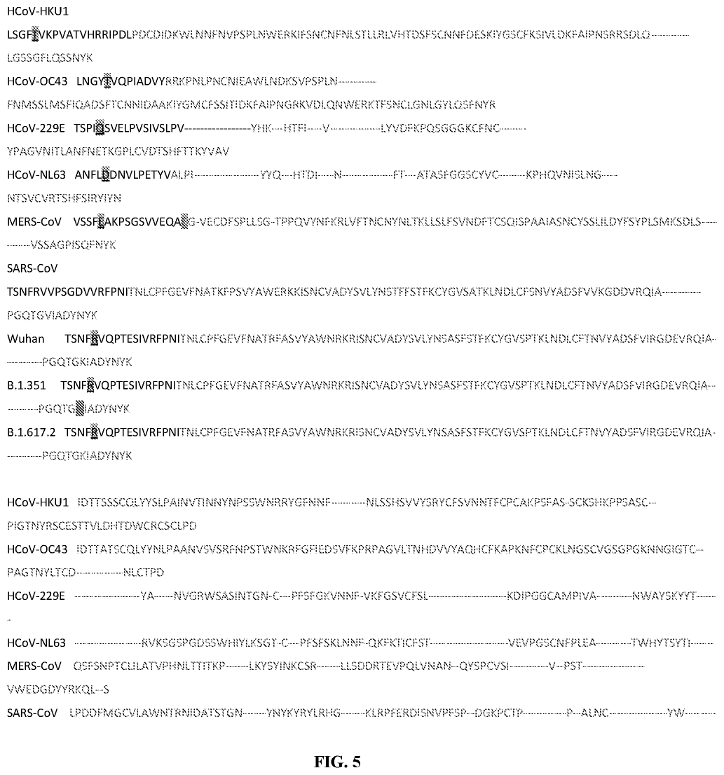

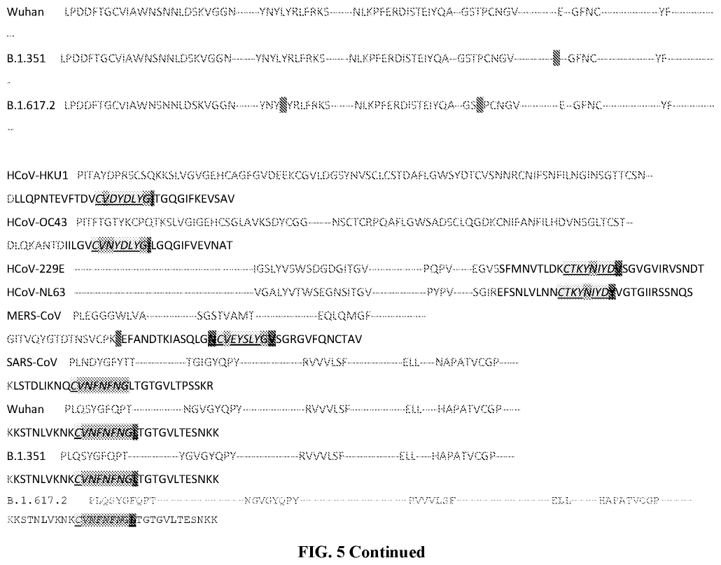

SARS-COV and MERS-COV RBDs recognize different receptors. SARS-CoV recognizes angiotensin-converting enzyme 2 (ACE2) as its receptor, whereas MERS-COV recognizes dipeptidyl peptidase 4 (DPP4) as its receptor. Like SARS-COV, SARS-COV-2 also recognizes ACE2 as its host receptor binding to viral S protein. The RBD is the most likely target for the development of virus attachment inhibitors, neutralizing antibodies, and vaccines. Based on the available crystal structures of the hCoVs the corresponding RBDs were analyzed to identify suitable frames (FIG. 5). The corresponding nucleotide and amino acid sequences for the tested vaccine antigens are provided above.

Expression and purification of selected RBD proteins

For the recombinant expression of RBDs the correct exposure of antigenic determinants has a significant impact on the immunogenicity—the antigen has to be correctly folded, contain not potentially antigenic non-vertebrate glycans, and, preferably, should have a glycosylation pattern similar to the native S protein. Based on these considerations HEK293 cell line was initially selected for antigen expression. Following non-satisfactory yields and purification issues, the decision was made to switch to the alternative XtenCHO cell line for antigen expression.

Expression of tag-free RBDs in XtenCHO cells. Each coronavirus Spike RBD antigen was produced using transient plasmid transfection of a serum-free suspension XtenCHO cell line. Initially small-scale expression tests were conducted. Purified plasmid from the respective antigen expression cassettes was transfected into the XtenCHO cell line. The cells are monitored for cell viability, cell density and cultured for up to 13 days (312 hours) while the protein antigens were secreted into the culture media. The Spike RBD antigens were purified through ion exchange chromatography (IEX) or affinity chromatography using RBD-specific antibodies developed by ProteoGenix. The research grade antigen was then buffer exchanged into the formulation buffer. An overview of the process is provided in FIG. 4.

For the scale-up of the target proteins, the ₚATX1 constructs were transfected in 1L HEK cells culture, and the culture medium samples were collected 13 days post transfection and used to run purification as established above. This new expression and purification strategy provided improved yields and sufficient to excellent purities for all RBDs tested (Table 1). The XtenCHO cells expression in combination with the IEX approach is scalable and applicable to GMP production phase. Additional process steps such as ion exchange chromatography will be implemented to enhance product purify.

TABLE 1
XtenCHO-expression results for use in a prime boost vaccine product.
Small scale expression was conducted in approx. 80 mL cell culture
media, scale-up expression in 1 L of cell culture media.

| Construct | Expression^{a} | Yield [mg] | Purity [ %] |
|---|---|---|---|
| SARS-CoV-2 Wuhan | Small-scale | 0.83 | >95 |
| | Scale-up | 16 | >90 |
| SARS-CoV-2 B.1.351 | Small-scale | 0.9 | >95 |
| | Scale-up | 15 | >90 |
| SARS-COV-2 B.1617.2 | Small-scale | 1.96 | >95 |
| | Scale-up | 21 | >90 |
| MERS-CoV | Small-scale | 5 | >80 |
| | Scale-up | 51 | >90 |
| SARS-CoV-2_B.1.1.529, | Small Scale | 0.06 | >80 |
| BA.1 | Scale up | 0.95 | >80 |

^{a}Small scale expression was conducted in approx. 80 mL cell culture media, scale-up expression in 1 L of cell culture media.

### Formulation of the Protein-boost Vaccine Component

The recombinant RBD proteins described above were formulated with aluminum hydroxide (Alum, MERS-RBD) or aluminum phosphate (all SARS-CoV-2 RBDs) hydrogel adjuvant. Briefly, 25 μg of RBD antigen in pH 7.4 saline buffer was mixed with 750 μg of 2% Alhydrogel (InvivoGen) and incubated with shaking for 30 min at room-temperature to allow adsorption, followed by storage at 2-8° C. until use. All vaccines were diluted in sterile saline before administration.

### Animal Studies

Across the project, the teams leverage the animal facilities at Tiba and turn to external vendors when necessary to complete animal studies that are beyond the in-house capacity. The study protocols were reviewed by Latham Biopharma Group, and external animal studies are conducted with their supervision.

### Results

### Preliminary Assessment of the Immunogenicity of RBD Protein Alum Formulations in C57BL/6 Mice Mouse

The primary objective of this study was to determine if selected RBD protein-Alum formulations would elicit an antibody response when injected intramuscularly (IM) into C57BL/6 mice and to test the effective dose range of the protein-alum formulation in vivo. Protein vaccines were administered as indicated in the Table below. Following dose administration on Day 1, blood was sampled and processed to serum at 2 weeks for analysis of anti-RBD antibody titers by ELISA.

| | | | | Route of | |
|---|---|---|---|---|---|
| | | Test | Dose | Adminis- | |
| Group | N | Material | (μg) | tration | Key Endpoint |
| 1 | 5 | SARS-CoV-2 | 15 | IM | Serum sampling for |
| 2 | (M) | Wuhan RBD | 3 | | ELISA analysis of |
| 3 | | | 0.6 | | titers for SARS- |
| 4 | | SARS-CoV-2 | 15 | | CoV-2 Wuhan and |
| 5 | | B.1.351 RBD | 3 | | B.1.351 variants. |
| 6 | | | 0.6 | | and MERS |
| 11 | | Negative | 0 | | |
| | | Control | | | |

To determine if the protein-Alum formulations would elicit an antibody response, as well as to test the effective dose range, 0.6, 3, or 15 μg of protein was injected into mice i.m. and serum was sampled 2 weeks later and analyzed by ELISA.

The results of the ELISAs (FIG. 1A-1C) show that all RBDs, when mixed with Alum and injected i.m. at 15 μg doses, generate detectable antibody responses to the corresponding RBD when assayed by direct ELISA. At a 15 μg dose, there was also significant cross-reactivity between the B.1.351 RBD and Wuhan RBD. In fact, antibody titers against the B.1.351 RBD were higher in mice vaccinated with Wuhan RBD than in mice vaccinated with B.1.351 RBD. One would expect that vaccination with a particular RBD would give the highest antibody titer against that same RBD, but it may be that the B.1.351 RBD protein is not as immunogenic as the Wuhan RBD, either due to its inherent immunogenicity or differences in the way the recombinant protein was designed, produced, or purified. In contrast, neither SARS-COV-2 variant RBD protein elicited a significant antibody response at 3 or 0.6 μg. No notable cross-reactivity between the MERS RBD and either of the SARS-COV-2 variant RBDs was observed.

The MERS RBD protein mixed with alum elicited an antibody response in all animals at both the 15 and 3 μg doses, but only showed a response in one animal at the 0.6 μg dose. In agreement with the RNA immunogenicity data, no cross-reactivity was seen between the MERS RBD and either of the SARS-CoV-2 variant RBDs. In addition, mice vaccinated with alum-formulated HKU1 RBD were sampled at the same time point, serum samples from these mice were tested in ELISAs against all other RBDs, although an antibody response against any of the three was not seen.

This experiment allowed a determination that alum formulations of all proteins produced as described in section 1.1.2 elicit dose-dependent antibody responses in C57BL/6 mice after 2 weeks at a high (15 μg) dose. While 3 and 0.6 μg of protein do not show equally strong antibody responses at week 2 in this experiment, it must be taken into account that the intended use of these proteins is in combination with an RNA prime, meaning the dose may not be needed to be as high to function as a boost.

To test this hypothesis, the potency of a protein RBD as a booster immunization was evaluated in a preliminary experiment in a cohort of mice injected 38 days prior with full-length COVID-19 variant B.1.351 Spike protein replicon RNA.

### Preliminary Assessment of the Immunogenicity of a Protein Booster Following Prime Immunization

The primary objective of this study was to determine the minimally immunogenic dose of a selected protein RBD booster following a prime immunization with a mRNA LNP encoding the SARS-COV-2 B.1.351 Spike protein replicon RNA in C57BL/6 mice as indicated in the Table below.

### Study Design for Assessment of Minimally Immunogenic Dose Following a Prime Immunization in C57BL/6 Mice

| | | RNA Prime | | Protein Boost | | | Key Endpoint |
|---|---|---|---|---|---|---|---|
| | | Injection | Prime Dose | Injection | Boost Dose | | (21 Days |
| Group | N (Sex) | (Day 1) | (μg) | (Day 39) | (μg) | RoA | post-boost) |
| 1 | 3 (M) | Full-length | 5 | Wuhan RBD | 15 | IM | Serum sampling |
| 2 | | SARS-CoV-2 | 5 | protein | 3 | | for ELISA |
| 3 | | B.1.351 Spike | 5 | | 0.6 | | analysis of titers |
| 4 | | protein | 5 | | 0 | | for SARS-CoV-2 |
| 5 | | replicon RNA | 0 | | 15 | | Wuhan |

The Wuhan RBD protein was prepared and injected as described above and serum from the boosted animals was analyzed 21 days later. Briefly. cohort of mice were immunized with full-length SARS-COV-2 variant B.1.351 Spike protein replicon RNA on Day 1 of the study (i.e., prime injection). Thirty-eight (38)-days post-prime injection, animals were administered a boost injection of the Wuhan RBD protein. Serum was collected from the animals 21-days post-boost injection for analysis. Specifically, antibody responses against the Wuhan RBD were measured by ELISA. In addition, semi-quantitative surrogate viral neutralizing titer (sVNT; GenScript catalogue number L00847; [Tan 2020]) was evaluated using the same samples (FIG. 2). nimals immunized with the B.1.351 variant on Day 1 (i.e., Prime only; Group 4) and with the Wuhan RBD booster on Day 39 (i.e., Boost only; Group 5) demonstrated an antibody response to the Wuhan variant. However, animals administered a protein boost (0.6, 3, or 15 μg of Wuhan RBD) following a prime immunization elicited an antibody response that was orders of magnitude higher than the prime only and/or boost only groups. In addition, the data indicate that a lower dose of the boost protein is required to elicit an equivocally immunogenic response in animals immunized with a mRNA LNP vaccine. Specifically, boost doses of 15 and 3 μg resulted in a similar titer while the 0.6 μg dose resulted in a slightly lower, but still similar, antibody response (FIG. 2).

### Non-Glp Assessment of the Inflammatory Response in C57BL/6-MICE-Initial Acute Toxicity Signals

The objective of this study was to compare systemic inflammatory response biomarkers induced by RNA lipid nanoparticles to those of RBD protein antigen formulated with aluminum adjuvant (Alhydrogel). The study design is shown in the Table below.

### Study Design for Assessment of Inflammation Response Following a Prime Immunization in C57BL/6 Mice

| | N | | Dose | | |
|---|---|---|---|---|---|
| Group | (Sex) | Injection | (μg) | RoA | Serum collection |
| 1 | 5 | Wuhan RBD protein | 15 | IM | 6 and 24 h after |
| 2 | (M) | B.1.351 RBD protein | 15 | | Injection |
| 3 | | MERS RBD protein | 15 | | |
| 4 | | Wuhan RBD protein | 3 | | |
| 5 | | B.1.351 RBD protein | 3 | | |
| 6 | | MERS RBD protein | 3 | | |
| 7 | | PBS | 0 | | |
| 8 | | RNA/LNP | 5 | | |

Current COVID-19 vaccines induce inflammatory responses that may be dose-limiting, and can lead to several days of fatigue, pain, chills, and fever after administration. To evaluate the level of inflammatory cytokine induction produced by the RNA delivery technology and protein vaccines under study here in comparison to an LNP benchmark control, BALB/c mice were injected with 5 μg formulated RNA or RBD protein at 15 or 3 μg doses. Serum concentrations of Interleukin 6 (IL-6), chemokine (C—X—C motif) ligand 1 (CXCL1), and regulated on activation, normal T cell expressed and secreted RANTES) protein, were measured at 6 and 24 h post-injection.

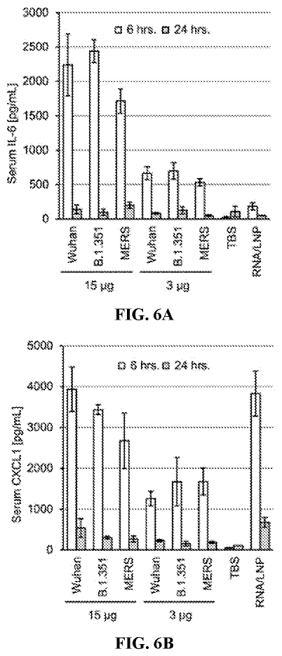

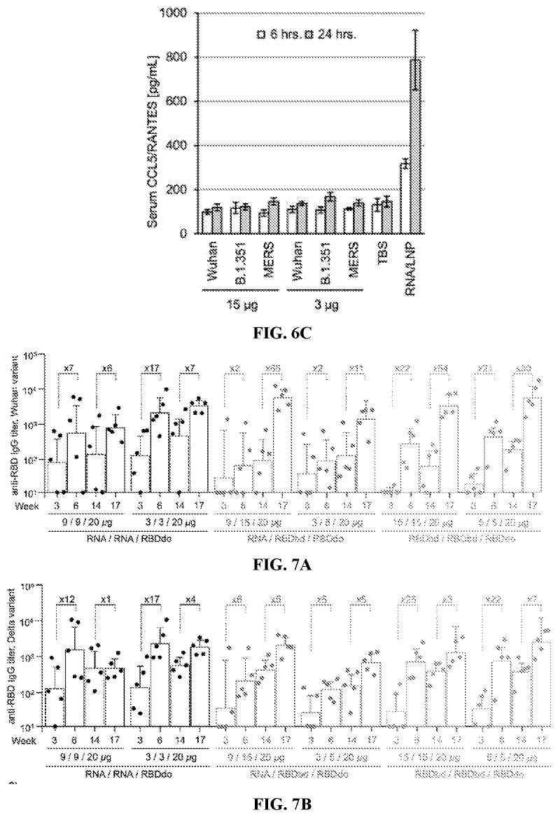

Interleukin 6 (IL-6) is expressed early in response to injury or infection, and triggers host defense mechanisms through the regulation of acute phase responses, hematopoiesis, the induction of fever, and innate and adaptive immune reactions. For initial toxicity studies, RNA was formulated in parallel with an exemplary cationic lipid-based LNP formulation to serve as a benchmark control. C57BL/6 mice (n=6) were immunized with the indicated vaccines at 15 or 3 μg dose levels for the RBD protein and 5 μg dose levels for the RNA/LNP formulation. Serum was collected 6 (white bars) and 24 hrs (grey bars) after the last injection. PBS injection served as negative control. Titers were determined by use of the R&D Systems DuoSet ELISA development system (Catalog numbers: IL-6: DY406, CXCL1: DY453, RANTES/CCL5: DY478). Serum volumes: IL-6:10 μL (1:5 dilution), CXCL1: 7 μL (1:6.25 dilution), RANTES/CCL5: 8 μL (1:7.14 dilution).Relative murine IL-6 levels are shown in FIG. 6A. Importantly, Tiba's formulation induced substantially less IL-6 stimulation than the LNP comparator, with IL-6 levels appearing to be negligible by 24 h post-injection. The protein vaccines were formulated as described above and tested similarly, with TBS vehicle injection serving as a negative control. While initial IL-6 induction was quite high for the tested RBDs (˜50× higher serum concentration compared to typical mouse serum baseline of ˜40 μg/ml), by 24 h the levels of IL-6 had waned to near equivalence with the control, suggesting transience and no systemic toxification.

The chemokine CXCL1 levels are shown in FIG. 6B. CXCL1 is an important chemoattractant for neutrophils and other non-hematopoietic cells to sites of injury or infection, and thus plays a dominant role in coordinating the molecular mechanisms underlying reactogenicity at vaccination sites (redness, swelling, etc.). Again, induction was observed for all materials at 6 h, however by 24 h responses had waned to baseline levels, with the highest residual signal observed in the LNP control group.

Finally, the concentration of serum RANTES was studied as an additional surrogate marker of leukocyte recruitment and activation (FIG. 6C). RANTES similarly plays a role in leukocyte recruitment to sites of established inflammation but is generally expressed at lower systemic levels than CXCL1. Then results showed that this was the case (FIG. 5B), with only the LNP benchmark inducing substantially higher levels at 6 h and 24 h compared to TBS baseline.

Collectively, these data indicate that the RBD antigen protein boost initially induces response of the immune system, which is required to trigger the processes required to establish immune protection. All markers studies waned for all protein RBD-formulation tested to near equivalence with the control within 24 hrs, suggesting absence of systemic toxification and potentially lower side effects than for the RNA/LNP formulation.

Altogether, an RNA payload has been selected that will express and drive immune responses against full-length B.1.351 Spike protein, and B.1.617.2/MERS RBDs. The recombinant Wuhan strain and MERS RBDs are confirmed to be effective when adjuvanted with alum. The preliminary cytokine analysis indicate that both the prime and boost vaccines as disclosed herein will exhibit significantly fewer side effects than a comparable LNP-based product.

### Immunogenicity Assessment of a RBD Booster Vaccine in Previously Prime-Vaccinated Syrian Golden Hamsters

The objective of this study was to evaluate the immunogenicity of different combinations prime-boost vaccines (RNA and/or Protein) administered via intramuscular injection to male and female Syrian Golden Hamsters. The RNA vaccine coded for the full-length spike protein of the Beta variant, while the protein formulation consisted of a 1:1 mixture of the RBDs of the Beta and Delta variants on alum. Antibody titers were assessed by ELISA against SARS-CoV-2 strains: Wuhan, Delta, and Omicron to assess the potency of serological responses.

Male and female Syrian gold hamsters (appropriately randomized) were administered a single prime injection on Day 1 and a booster injection on Day 22 as indicated in the Table below.

### Study Design

| | | Prime Injection | Dose | Boost Injection | Dose |
|---|---|---|---|---|---|
| Group | N | (Day 1) | (μg) | (Day 22) | (μg) |
| 6 | 5 | LNP Control | 0 | LNP Control | 0 |
| 7 | (2M/3F) | RNA beta - Full Lengthi Spike | 9 | RNA beta - Full Length Spike | 9 |
| 8 | | RNA beta - Full Length Spike | 9 | Protem - Beta & Delta RBD | 15 |
| 9 | | Protein - Beta & Delta RBD | 15 | Protein - Beta & Delta RED | 15 |
| 10 | | RNA beta - Full Length Spike | 3 | RNA beta - Full Length Spike | 3 |
| 11 | | RNA beta - Full Length Spike | 3 | Protem - Beta & Delta RBD | 5 |
| 12 | | Protein - Beta & Delta RBD | 5 | Protein - Beta & Delta RBD | 5 |

Endpoints evaluated included mortality, clinical observations, injection site observations (modified Draize Scoring), body weights, body temperature, and blood collection for assessment of immunogenicity. Blood was collected on Day 1 (prior to prime injection), 24-h post prime injection, Day 15, Day 22 (prior to booster injection), Day 29, Day 36, and Day 45. Blood samples were processed to serum and the serum samples were analyzed using an ELISA-based method to generate endpoint titers against a selection of SARS-COV-2 RBD proteins.

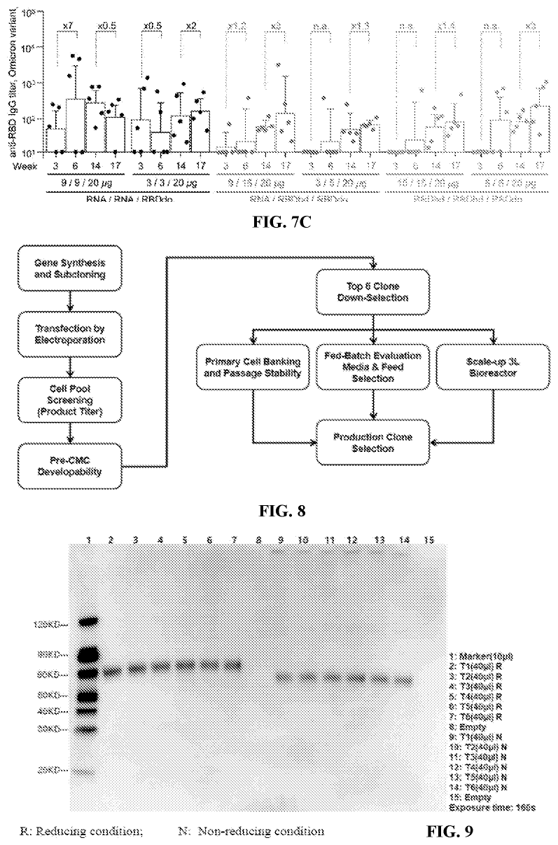

The strongest enhancement of anti-Wuhan and Delta antibody levels from prime (FIG. 7, week 3 data) and boost (FIG. 17, week 6 data) is observed for the RBD/RBD combination (21 to 25-fold enhancement), followed by the RNA/RNA combination (7- to 17-fold enhancement) and the RNA/RBD combination 2 to 6-fold enhancement), with no significant differences between the high and the low dose groups. Yet, when comparing RNA/RNA and RNA/RBD data, the RNA prime induced an in average significantly better response for the first than for the latter group, which indicates strong animal to animal variations for this animal model. Anti-Omicron antibody levels were found to be reduced by over one order of magnitude for all groups and dose levels, with a non-detectable prime response for the RBD-prime groups.

From a safety standpoint, all animals survived to their scheduled sacrifice dates. In addition, there was also no evidence of adverse reactions by monitoring of clinical signs, differences in body weight, or monitoring of the injection site.

Collectively, these data indicate that the booster injection of RBD formulated with alum elicited an immunogenic response under both regimens tested with a significant enhancement of titers of up to 25-fold, which was comparable or higher than the RNA/RNA standard, without indications of any adverse effects.

### Follow-Up Immunogenicity Assessment of a Booster Vaccine in Previously Fully Vaccinated Syrian Golden Hamsters

The objective of this study was to evaluate the immunogenicity of alum formulated RBD-protein dedicated booster vaccine administered via intramuscular injections in male and female Syrian Golden hamsters, which were previously prime and boost vaccinated in the studies above, represented in FIGS. 7A-C. The protein formulation consisted of the RBDs of a 1:1 mixture of the Delta and the Omicron (BA.1) variants formulated with alum. Antibody titers were assessed by ELISA against SARS-COV-2 strains: Wuhan, Delta, and Omicron to assess the potency of serological responses.

Previously vaccinated male and female Syrian gold hamsters were administered a single intramuscular injection on Day 1 (Day 99 after prime injection) as indicated in the Table below.

Study Design

| Group | N | Day 1 Prime Injection | Dose (μg) |
|---|---|---|---|
| 7 | 5 (2M/3F) | Protein Delta/Omicron RBD | 20 |
| 8 | | Protein Delta/Omicron RBD | 20 |
| 9 | | Protein Delta/Omicron RBD | 20 |
| 10 | | Protein Delta/Omicron RBD | 20 |
| 11 | | Protein Delta/Omicron RBD | 20 |
| 12 | | Protein Delta/Omicron RBD | 20 |

Evaluated endpoints include mortality, clinical observations, injection site observations (modified Draize Scoring), body weights, body temperature, and blood collection for assessment of immunogenicity. Blood was collected on Day 1 (prior to prime injection), Day 7, Day 15 and Day 22. Blood samples were processed to serum and the serum samples were analyzed using an ELISA-based method to generate endpoint titers against a selection of SARS-COV-2 RBD proteins.

In general, titer elevation is most pronounced for the Wuhan variant, followed by Delta and Omicron. A significant enhancement of antibody levels (FIG. 7A-C, week 17 data) is observed for all pre-vaccinated groups, with a similar response pattern for all previously RNA/RBD or RBD/RBD vaccinated groups (up to 65-fold enhancement), with a less pronounced effect for the RNA/RNA pre-vaccinated groups (up to 7-fold enhancement). During the 11 week break between the first boost (week 3 previous study) and the second boost in this study (FIG. 7A-C, week 14 data), determined antibody titers dropped significantly, roughly one order of magnitude for the homologous regimen groups (RNA/RNA and RBD/RBD), while for the heterologous regimen (RNA/RBD) a further increase of titers was observed, which may be indicative of prolonged immune maturation process in the heterologous regimen.

From a safety standpoint, all animals survived to their scheduled sacrifice dates. In addition, there was no evidence of adverse reactions by monitoring of clinical signs, body weight, and injection sites.

Collectively, these data indicate that the booster injection of RBD on alum elicit an immunogenic response for all three-regimen tested with a significant enhancement of titers of up to 65-fold, without indications of any safety relevant adverse effects.

### Example 2: Preparation of Kv-0620: A Novel Protein Booster Vaccine for the Prevention of Coronavirus Disease

These studies provide a protein subunit booster vaccine candidate (KV-0620), which is based on the receptor binding domain (RBD) of the SARS-COV-2 Spike(S) glycoprotein (residues R319-K537), which is responsible for mediating cell entry and interaction with host receptor angiotensin-converting enzyme 2 (ACE2). The RBD is the primary target of neutralizing antibodies. The RBD (residues R319-K537) protein subunit booster vaccine candidate is distinctly designed as a heterologous fusion dimer of the SARS-COV-2 variants of concern. Specifically, KV-0620 is a combination of the RBD (residues R319-K537) of SARS-COV-2 Delta (B.1.617.2) and RBD (residues R319-K537) of SARS-COV-2 Omicron (B.1.1.529, BA.1). The KV-0620 booster vaccine design addresses the continuing SARS-COV-2 antigen shift through incorporation sequences of both serotype 1 and 2. A vaccine combining elements of any current omicron sub-lineage with a complementary lineage from the original serotype should induce effective protection.

### KV-0620 Composition (Chemical Name and Structure)

KV-0620 is a heterologous fusion dimer consisting of a combination of the RBD (residues R319-K537) of SARS-COV-2 Delta (B.1.617.2) and RBD (residues R319-K537) of SARS-COV-2 Omicron (B.1.1.529, BA.1).

The KV-0620 antigen Drug Substance was produced via mammalian expression in Chinese Hamster Ovary (CHO) cells. The final drug product (DP) is composed of a 75 μg of the heterologous fusion dimer, and 850 μg of aluminum per 0.5 mL injection.

The KV-0620 vaccine is comprised of purified recombinant protein antigen formulated with aluminum adjuvant. The recombinant KV-0620 antigen is expressed in a stable Chinese Hamster Ovary (CHO) cell line cultured under serum-free conditions. The antigens are secreted into and purified from the culture medium. A description of the generation of the raw materials, manufacturing process and quality testing and specifications for drug substance and drug product are described in further detail below.

The KV-0620 vaccine antigen is a purified, ˜50 kDa recombinant protein that is a heterologous fusion dimer of RBD (residues R319-K537) of the SARS-CoV-2 Delta (B.1.617.2) variant and the RBD (residues R319-K537) of SARS-CoV-2 Omicron (B.1.1.529,BA.1) variant. Characteristics of the KV-0620 antigen are provided in Table 2. A description of KV-0620 recombinant protein antigen production in CHOK1-GenS cells following stable cell line development is outlined in the following subsections.

TABLE 2
Characteristics of KV-0620

| Product | Peptide | Molecular | Isoelectric | Charge in PBS |
|---|---|---|---|---|
| Name | Length | Mass [kDa] | Point ^{a} | buffer (pH = 7.37) ^{a} |
| KV-0620 | 438 aa | 49.61 | 8.5 | 16.3 |

^{a} Isoelectric point and Charge predicted [Prot Pi] Protein Tool n.d.]

### KV-0620 Gene Sequence and Expression Plasmid Generation

The DNA coding sequence (SEQ ID NO:17) of the KV-0620 antigen was codon optimized for expression in CHOK1-GenS cells and synthesized by artificial gene synthesis by GenScript.

(SEQ ID NO: 17)
GGCGCGCCGCCGCCACCATGGGCTGGTCCTGCATCATCCTGTTTC
TGGTGGCTACCGTACCGGCGTGCACTCTAGAGTGCAGCCTACCGA
GTCTATCGTGCGGTTCCCCAACACACCAACCTGTGTCCTTTCGGC
GAGGTGTTCAACGCCACCAGATTCGCCTCTGTGTACGCCTGGAAC
CGGAAGCGGATCTCTAACTGCGTGGCCGACTACTCCGTGCTGTAC
AACTCCGCCTCCTTCAGCACCTTCAAGTGCTACGGCGTGTCCCCT
ACCAAGCTGAACGACCTGTGCTTCACCAACGTGTACGCCGACTCC
TTCGTGATCAGAGGCGACGAAGTGCGGCAGATCGCTCCTGGACAG
ACCGGCAAGATCGCCGACTACAACTACAAGCTGCCCGACGACTTC
ACCGGCTGTGTGATCGCTTGGAACTCCAACAACCTGGACTCCAAA
GTCGGCGGCAACTACAATTACCGGTACAGACTGTTCCGGAAGTCC
AACCTGAAGCCTTTCGAGCGGGACATCTCCACCGAGATCTACCAG
GCTGGCTCCAAGCCTTGCAATGGCGTGGAAGGCTTCAACTGCTAC
TTCCCACTGCAGTCCTACGGCTTCCAGCCTACAAACGGCGTGGGC
TACCAGCCTTACAGAGTGGTGGTGCTGTCCTTCGAGCTGCTGCAT
GCTCCTGCTACCGTGTGCGGCCCTAAGAAATCTACCAACCTGGTC
AAGAACAAGCGGGTGCAGCCCACTGAGAGCATTGTGCGCTTCCCT
AATATCACAAATCTGTGCCCCTTTGACGAAGTCTTTAATGCTACC
CGCTTCGCTTCCGTGTATGCTTGGAATAGAAAGCGGATCAGCAAT
TGCGTCGCCGATTACAGCGTCCTGTACAATCTGGCCCCATTCTTC
ACCTTTAAGTGTTATGGCGTCAGCCCCACCAAGCTCAATGATCTC
TGTTTTACCAATGTCTACGCCGATAGCTTTGTGATTCGCGGAGAT
GAAGTCCGCCAGATCGCACCAGGCCAGACTGGAAATATCGCTGAT
TACAATTATAAGCTCCCTGATGATTTCACAGGATGCGTTATCGCC
TGGAATAGCAACAAGCTGGACAGCAAGGTGTCCGGCAATTACAAC
TACCTGTATCGGCTGTTCCGCAAGAGTAATCTGAAGCCCTTTGAG
AGAGACATCAGTACAGAAATCTATCAGGCCGGCAACAAGCCCTGT
AACGGCGTCGCAGGCTTTAACTGTTATTTTCCCCTGCGCTCCTAC
TCCTTCCGGCCTACTTATGGCGTGGGCCATCAACCATATCGCGTG
GTGGTTCTGAGTTTCGAACTCCTGCACGCCCCAGCCACAGTGTGT
GGCCCCAAAAAGAGCACCAATCTCGTTAAGAACAAGTAGTGAGGC
CGGCC

The synthesized KV-0620 gene sequence was then cloned into plasmid pGenHT1.0-DGV to create pGenHT1.0-DGV RBD_SARS-COV-2_hcDimer_(R519-K537) (Plasmid ID: C7885HD010-2) for expression in mammalian cells. Also included was a Kozak sequence immediately upstream of the start codon (SEQ ID NO:17, residues in bold, italicized) and a mammalian signal sequence was also incorporated (SEQ ID NO:17, underlined sequence) to drive secretion of the KV-0620 antigen into the culture medium. The encoded and antigen amino acid sequences are provided in SEQ ID NO:15 and SEQ ID NO: 16 respectively.

The history of the parent pGenHT1.0-DGV plasmid is fully traceable and registered with a Drug Master File (DMF) which will be cross-referenced as part of the IND submission. The KV-0620 expression vectors used for transfections were verified by DNA sequencing before stable transfection and clone selection.

Coded Amino Acid Sequence of the KV-0620
Heterodimeric Antigen is shown below.
(SEQ ID NO: 15)
MGWSCIILFLVATATGVHSRVQPTESIVRFPNITNLCPFGEVENA
TRFASVYAWNRKRISNCVADYSVLYNSASFSTFKCYGVSPTKLND
LCFTNVYADSFVIRGDEVRQIAPGQTGKIADYNYKLPDDFTGCVI
AWNSNNLDSKVGGNYNYRYRLFRKSNLKPFERDISTEIYQAGSKP
CNGVEGENCYFPLQSYGFQPTNGVGYQPYRVVVLSFELLHAPATV
CGPKKSTNLVKNKRVQPTESIVRFPNITNLCPFDEVFNATRFASV
YAWNRKRISNCVADYSVLYNLAPFFTFKCYGVSPTKLNDLCFTNV
YADSFVIRGDEVRQIAPGQTGNIADYNYKLPDDFTGCVIAWNSNK
LDSKVSGNYNYLYRLFRKSNLKPFERDISTEIYQAGNKPCNGVAG
ENCYFPLRSYSFRPTYGVGHQPYRVVVLSFELLHAPATVCGPKKS
TNLVKNK.
Mature Amino Acid Sequence of KV-0620
Heterodimeric Antigen is shown below.
(SEQ ID NO: 16)
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGENCYFPLQSYGFQ
PTNGVGYQPYRVVVLSFELLHAPATVCGPKKSTNLVKNKRVQPTE
SIVRFPNITNLCPFDEVFNATRFASVYAWNRKRISNCVADYSVLY
NLAPFFTFKCYGVSPTKLNDLCFTNVYADSFVIRGDEVRQIAPGQ
TGNIADYNYKLPDDFTGCVIAWNSNKLDSKVSGNYNYLYRLFRKS
NLKPFERDISTEIYQAGNKPCNGVAGFNCYFPLRSYSFRPTYGVG
HQPYRVVVLSFELLHAPATVCGPKKSTNLVKNK.

### Stable Cell Line Development for KV-0620 Production and GMP Readiness

The selection of the stable cell line for KV-0620 production leveraged and existing and high throughput cell line generation, screening and development platform and workflow at the manufacturer. The steps involved with this process are outlined in FIG. 7.

### Stable Cell Pool Screening

The expression vector pGenHT1.0-DGV RBD_SARS-CoV2_hcDimer_(R519-K537) was transfection grade plasmid prepared and then electro-transfected into 6 independent parental CHO-K1 host cells. In this process, the target gene integrates into the host cell chromosome and expresses the target product. As outlined in FIG. 8, six independent transfections were performed and the expression of the KV-0620 antigen assessed in these initial cell pools.

At 48 hours post-transfection the expression level of the KV-0620 antigen was qualitatively examined by Western blotting using a SARS-COV-2 Omicron-reactive Spike monoclonal antibody (Sino Biological, Cat: 40592-MM117) as shown in FIG. 9 (reducing conditions left lanes, non-educing conditions in the right lanes) from culture supernatant. In addition, the amount of the KV-0620 was determined from the 6 individual transfected cultures by quantitative ELISA assays (Table 3). Both methodologies confirm that the KV-0620 antigen expression was consistent across all cultures.

TABLE 3
Expression Level of KV-0620 at 48 Hours Post-Transfection^{a}

| Transfected Cell Number | Dilution | Titer (mg/L] |
|---|---|---|
| T1 | 1000 | 0.24 |
| T2 | 1000 | 0.22 |
| T3 | 1000 | 0.20 |
| T4 | 1000 | 0.21 |
| T5 | 1000 | 0.19 |
| T6 | 1000 | 0.20 |

^{a}SARS-CoV-2 Delta (B.1.617.2) variant Spike RBD ELISA Kit, Cat: KIT40592b

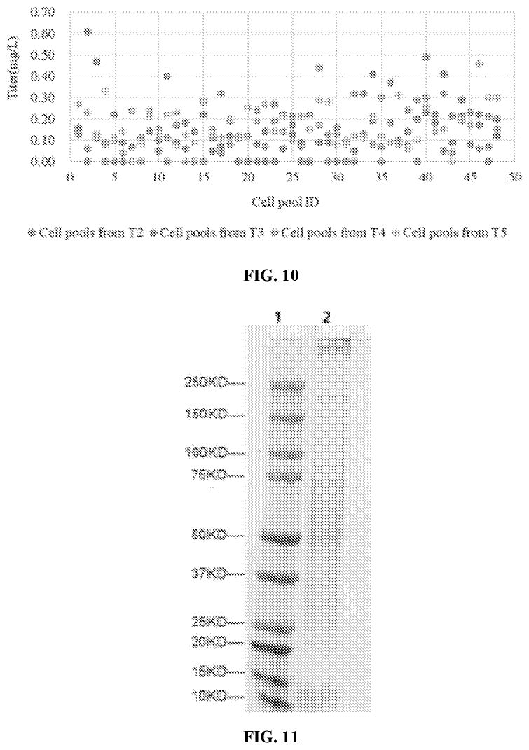

KV-0620 was successfully expressed in the 6 parallel transfections and qualitative confirmed by Western blot and ELISA. At 48 hours post-transfection, the transfected cells of T2, T3, T4 and T5 were seeded into 24-well plates for cell pool screening with the selection medium (CD CHO medium+25 μM methionine sulfoximine (MSX) plus anti-clumping agent (ACA, 400x) at the density of 0.08×10⁶ cells/well. The seeding day was defined as day 0. Cells were subcultured with selection medium every 4-6 days. After screening for 19 days, the conditioned media from a 6-day culture of pools were collected for ELISA analysis (FIG. 10).

KV-0620 was successfully expressed in CHOK1-GenS with the highest expression level of 0.61 mg/L (ELISA) at cell pool screening in 24-well plates.

### Cell Clone Screening

The fast cell pool screening was done in basic CD CHO+25 μM MSX+(400×) ACA medium. Inoculation on Day 1 was done with 0.40E+06 cells/mL in a volume of 3.0 mL. The culture conditions were 37.0° C., 5.0% CO2, and 100 rpm. Subsequently the culture was grown for 6 days and then harvested. The supernatant was harvested for qualitative analysis by ELISA as indicated in Table 4.

TABLE 4
Expression level of KV-0620 after 6 Days Post-Transfection^{a}

| Construct | Dilution | Titer [mg/L] |
|---|---|---|
| T6 | 5000 | 0.90 |

^{a}SARS-CoV-2 Delta (B.1.617.2) variant Spike RBD ELISA Kit, Cat: KIT40592b

KV-0620 was successfully expressed in CHOK1-GenS with the expression level of 0.90 mg/L (FIG. 11, Table 4) at fast cell pool batch. Next, single clone screening was conducted using limited dilution cloning method. Seeding density is <0.5 cell/well. A total of 40 96-well plates are used for this screening process. Monoclonality confirmation was conducted by clone imaging based on images captured by Cell Metric™ CLD. On day 0, 1, 2, 7, and a later day with adequate cell confluency of 30%. After 3-4 weeks, the expression level of single cell clones was examined by a titer determination method, ELISA. The top high-producing clones were confirmed via monoclonality to assess their expression levels by fed-batch culture evaluation.

### Primary Cell Bank (PCB)

The top 6 cell clones that were confirmed to be mycoplasma free and then selected for cell expansion. The desired cells were cultured in shake flasks, harvested and frozen as PCB (with 10 vials/clone). The viability and recovery of frozen cells were verified. Six (6) PCB clones were recovered from cryopreservation and assess cell viability in MSX selection medium. Passages were made every 3 days. Cell density and cell viability were examined when passing the cells. Passage 14, 17 and 20 of cells (5-6×10⁶ cells/vial, 3 vials) were cryopreserved for fed-batch evaluation.

Based on cell growth performance during 20 passages, the top 3 clones were selected and recovered for analysis (passage 0, 17 and 20) of genetic stability (sequencing of target gene sequence) and performance in for fed-batch culture (passage 0 and 20) as measured by cell density, viability and product titer.

KV-0620 is useful for the prevention of SARS-COV-2 infection. Liquid formulation include a single-dose vial with 0.5 mL administered per injection: 75 μg of antigen

### REFERENCES

1. Excler, et al. Nature Medicine 2021, 27 (4), 591-600.
2. Shaw, et al. Lancet 2021, 397 (10289), 2043-2046.
3. He, et al. Emerging Microbes & Infections 2021, 10 (1), 629-637.
4. Krammer, Nature 2020, 586 (7830), 516-527.
5. Chakraborty, et al. Advanced Drug Delivery Reviews 2021, 172, 314-338.
6. Kustin, et al. Nature Medicine 2021, 27 (8), 1379-1384.
7. Juthani, P. V.; Gupta, A.; Borges, K. A.; Price, C. C.; Lee, A. I.; Won, C. H.; Chun, H. J., Hospitalisation among vaccine breakthrough COVID-19 infections. The Lancet Infectious Diseases. doi: 10.1016/S1473-3099 (21) 00558-2 (2021).
8. Shastri, et al. Frontiers in Medicine 2021, 8 (1379). Xu, J. et al. Antibodies and vaccines against Middle East respiratory syndrome coronavirus. Emerg Microbes Infect 8, 841-856, doi: 10.1080/22221751.2019.1624482 (2019).
9. Teo, SP Review of COVID-19 mRNA Vaccines: BNT162b2 and mRNA-1273. Journal of Pharmacy Practice. April 2021. doi: 10.1177/08971900211009650
10. Pfizer. Pfizer and BioBTech choose lead mRNA vaccine candidate against COVID-19 and commence pivotal phase 2/3 global study. News Press Release. https://www.pfizer.com/news/press-release/press-release-detail/pfizer-and-biontech-choose-lead-mrna-vaccine-candidate-0. Published Jul. 27, 2020.
11. Abdul-Rasool S, Fielding BC. Understanding Human Coronavirus HCoV-NL63. Open Virol J. 2010; 4 (1): 76-84. doi: 10.2174/1874357901004010076.
12. Pene F, Merlat A, Vabret A, et al. Coronavirus 229E-Related Pneumonia in Immunocompromised Patients. Clin Infect Dis. 2003; 37 (7): 929-932. doi: 10.1086/377612
13. Vassilara F, Spyridaki A, Pothitos G, Deliveliotou A, Papadopoulos A. A Rare Case of Human Coronavirus 229E Associated with Acute Respiratory Distress Syndrome in a Healthy Adult. Case Rep Infect Dis. 2018; 2018:1-4. doi: 10.1155/2018/6796839
14. Chahal J S, Khan O F, Cooper C L, et al. Dendrimer-RNA nanoparticles generate protective immunity against lethal Ebola, H1N1 influenza, and Toxoplasma gondii challenges with a single dose. Proc Natl Acad Sci. 2016; 113 (29): E4133-E4142. doi: 10.1073/pnas.1600299113
15. Janaszewska A, Lazniewska J, Trzepiński P, Marcinkowska M, Klajnert-Maculewicz B. Cytotoxicity of Dendrimers. Biomolecules. 2019; 9 (8): 330. https://doi.org/10.3390/biom9080330

1. A composition for boosting protecting a subject against a betacoronavirus disease comprising an effective amount of a fusion protein/peptide represented by the general formula:
RBD₁-L1-RBD₂-L2-RBDₙ, Formula I
where RBD₁ represents a first RBD sequence of a betacoronavirus, RBD₂ represents a second RBD sequence of a betacoronavirus, n is an integer represented the number of a subsequent RBD sequence(s) and L1 and L2 are optional first and second linkers, respectively and an adjuvant, and optionally, a pharmaceutically acceptable carrier.

2. The composition of claim 1, wherein the betacoronavirus is selected from the group consisting of SARS-COV, MERS-Cov and SARS-COV-2 viruses.

3. The composition of claim 2, wherein the betacoronavirus is a variant of SARS-COV-2, such as SARS-COV-2 B.1.1.7 (Alpha variant), SARS-COV-2 B.1.351 (Beta variant), SARS-COV-2 P.1 (Gamma variant), SARS-COV-2 B.1.617, SARS-COV-2 B.1.617.1 (Kappa variant), SARS-COV-2 B.1.621 (Mu variant), SARS-CoV-2 B.1.617.2 (Delta variant), SARS-COV-2 B.1.617.3, and SARS-COV-2 B.1.1.529 (Omicron variant); optionally, where n is 0, 1, 2, 3, 4 or 5.

4. (canceled)

5. The composition of claim 1, wherein: (a) the RDB sequence comprises SEQ ID NO: 2, 4, 6, 18, 19 or 20 or a functional variant thereof having more than 90%, 91%, 92%, 93%, 94%, 95%, 96%, 97%, 98%, or 99% identical to the amino acid sequence of SEQ ID NOs. 2, 4, 6, 18, 19 or 20; or (b) the RDB sequence comprises SEQ ID NO: 8, 10, 12, 15, 16, 21, 22 or 23 or a functional variant thereof having more than 90%, 91%, 92%, 93%, 94%, 95%, 96%, 97%, 98%, or 99% identical to the amino acid sequence of SEQ ID NOs 8, 10, 12, 15 and 16.

6. (canceled)

7. The composition of claim 7, (a) comprising SEQ ID NO:16; (b) wherein n=0; (c) wherein n=0; and/or (d) wherein the adjuvant is an alum or an alum derivative; optionally, wherein the adjuvant is an alum derivative comprising an aluminum hydroxide/oxyghydride gel or aluminum phosphate gel.

8. (canceled)

9. (canceled)

10. (canceled)

11. (canceled)

12. The composition of claim 7, comprising a fusion peptide 90%, 91%, 92%, 93%, 94%, 95%, 96%, 97%, 98%, or 99% identical to the amino acid sequence of SEQ ID NO: 16; optionally, wherein the fusion protein/peptide is fused with an adjuvanting protein, an immune-stimulating protein, or a peptide moiety.

13. (canceled)

14. The composition of claim 13, wherein the adjuvanting protein comprises a compound selected from the group consisting of: a keyhole limpet hemocyanin (KLH), and Concholepas hemocyanin (CCH).

15. The composition of claim 13, wherein the peptide moiety comprises a CD4+T cell-activating helper peptide.

16. The composition of claim 1, wherein the fusion protein/peptide is engineered in such a way so as to be arrayed in 3D space in a regular, repeated fashion in order to promote B cell receptor engagement, clustering, and activation.

17. The composition of claim 1, wherein fusion protein/peptide is arrayed or presented on a suitable carrier through electrostatic attraction selected from the group consisting of electrostatic immobilization of an antigen with a positive charge in the applied buffer or with a genetically fused tag coding for a highly basic peptide sequence on a negatively charged carrier and electrostatic immobilization of an antigen with a negative charge in the applied buffer or with a genetically fused tag coding for an acidic peptide sequence on a positively charged carrier.

18. The composition of claim 17, wherein the negatively charged carrier is selected from the group consisting of an anionic liposome, dendrimer, polynucleotide or synthetic nanoparticle).

19. The composition of claim 17, wherein the positively charged carrier is selected from the group consisting of cationic liposome, dendrimer or synthetic nanoparticle.

20. The composition of wherein the delivery vehicle is a dendrimeric particle.

21. A method of re-focusing the immune response of a subject against one or more pathogens comprising administering to the subject the composition of claim 1, wherein the subject was previously infected by the pathogen or vaccinated against the one or more pathogens.

22. The method of claim 21, wherein the pathogen is a betacoronavirus.

23. The method of claim 21, wherein the betacoronavirus is SARS-COV-2 or MERS CoV; optionally, wherein the betacoronavirus is a variant of SARS-CoV-2, such as SARS-COV-2 B.1.1.7 (Alpha variant), SARS-COV-2 B.1.351 (Beta variant), SARS-COV-2 P.1 (Gamma variant), SARS-COV-2 B.1.617, SARS-COV-2 B.1.617.1 (Kappa variant), SARS-COV-2 B.1.621 (Mu variant), SARS-COV-2 B.1.617.2 (Delta variant), SARS-CoV-2 B.1.617.3, and SARS-COV-2 B.1.1.529 (Omicron variant).

24. (canceled)

25. The method of claim 24, wherein: (a) the composition comprises SEQ ID NO:16; (b) n=0; (c) L1 and L2 are absent; and/or (d) the adjuvant is an alum or an alum derivative; optionally, wherein the adjuvant is an alum derivative comprising an aluminum hydroxide/oxyghydride gel or aluminum phosphate gel.

26. (canceled)

27. (canceled)

28. (canceled)

29. (canceled)

30. The composition of claim 21, comprising a fusion peptide 90%, 91%, 92%, 93%, 94%, 95%, 96%, 97%, 98%, or 99% identical to the amino acid sequence of SEQ ID NO: 16.

31. The method of claim 21, wherein the composition is administered by intranasally or by intramuscular injection.

32. The method of any claim 21, wherein the composition is administered to the subject in an amount effective to elicit a neutralizing antibody response against the one or more pathogens.


SEQUENCE LISTING (29 sequences)

SEQ ID NO: 1 - DNA, 794 bp, synthetic construct
  source 1..794: mol_type = other DNA
gaattcgccgccaccatgtacaggatgcagctgctgtcctgcatcgccctgagcctggccctggtgacaaattccgaggccaagcctagcggcagcgtggtggagcaggccgagggagtggagtgcgacttctcccccctgctgagcggcacccccccacaagtgtacaacttcaagagactggtgttcacaaactgtaattacaacctgaccaagctgctgtccctgttctccgtgaatgatttcacctgcagccagatctcccctgccgccatcgcctccaactgctactccagcctgatcctggattacttctcctaccccctgagcatgaagtccgatctgagcgtgagctccgccggccccatcagccagttcaactacaagcagtccttctccaaccctacctgtctgatcctggccaccgtgccccacaatctgaccaccatcaccaagcccctgaagtactcctacatcaataagtgtagcaggctgctgtccgacgatagaacagaggtgcctcagctggtgaatgccaaccagtacagcccctgcgtgagcatcgtgcctagcaccgtgtgggaggatggcgactactacaggaagcagctgtcccccctggagggcggcggatggctggttgcttccggcagcaccgtggccatgaccgagcagctgcagatgggcttcggcatcaccgtgcagtacggcacagacaccaatagcgtgtgtcctaagctggagttcgccaacgatacaaagatcgccagccagctgggcaattgagcggccgc

SEQ ID NO: 2 - protein, 256 aa, synthetic construct
MYRMQLLSCIALSLALVTNSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGN

SEQ ID NO: 3 - DNA, 722 bp, synthetic construct
  source 1..722: mol_type = other DNA
gaattcgccgccaccatgtacaggatgcagctgctgagctgtatcgccctgagcctggccctggtgaccaatagccaggccgagggcgtggagtgtgacttttcccctctgctgagcggcacccctcctcaggtgtacaatttcaagagactggtgttcacaaactgcaattacaacctgacaaagctgctgagcctgttctccgtgaatgacttcacatgcagccagatcagccccgccgccatcgccagcaactgctactcctccctgatcctggactacttctcctaccctctgtccatgaagagcgatctgagcgtgtccagcgccggccccatctcccagtttaactacaagcagtccttcagcaatcctacatgcctgatcctggccacagtgccccacaatctgaccaccatcaccaagcccctgaagtacagctacatcaacaagtgctccagactgctgagcgacgataggaccgaggtgcctcagctggtgaatgccaatcagtactccccttgtgtgtccatcgtgccttccacagtgtgggaggacggcgactactacaggaagcagctgtcccctctggagggcggcggctggctggttgctagcggatccaccgtggccatgaccgagcagctgcagatgggcttcggcatcacagtgcagtacggcacagacaccaacagcgtgtgtcccaagctgtgagcggccgc

SEQ ID NO: 4 - protein, 232 aa, synthetic construct
MYRMQLLSCIALSLALVTNSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 5 - DNA, 1502 bp, synthetic construct
  source 1..1502: mol_type = other DNA
gaattcgccgccaccatgtacaggatgcagctgctgtcctgcatcgccctgagcctggccctggtgacaaattccgaggccaagcctagcggcagcgtggtggagcaggccgagggagtggagtgcgacttctcccccctgctgagcggcacccccccacaagtgtacaacttcaagagactggtgttcacaaactgtaattacaacctgaccaagctgctgtccctgttctccgtgaatgatttcacctgcagccagatctcccctgccgccatcgcctccaactgctactccagcctgatcctggattacttctcctaccccctgagcatgaagtccgatctgagcgtgagctccgccggccccatcagccagttcaactacaagcagtccttctccaaccctacctgtctgatcctggccaccgtgccccacaatctgaccaccatcaccaagcccctgaagtactcctacatcaataagtgtagcaggctgctgtccgacgatagaacagaggtgcctcagctggtgaatgccaaccagtacagcccctgcgtgagcatcgtgcctagcaccgtgtgggaggatggcgactactacaggaagcagctgtcccccctggagggcggcggatggctggttgcttccggcagcaccgtggccatgaccgagcagctgcagatgggcttcggcatcaccgtgcagtacggcacagacaccaatagcgtgtgtcctaagctggagttcgccaacgatacaaagatcgccagccagctgggcaatgaggccaagccctccggctccgtggtggagcaagccgagggcgtggagtgtgacttctcccctctgctgagcggaacccctcctcaggtgtacaactttaagagactggtcttcaccaactgcaactacaatctgacaaagctgctgagcctgttcagcgtgaacgacttcacctgtagccagatcagccccgccgccatcgctagcaactgttactcctccctgatcctggactacttttcctaccccctctccatgaagtccgacctgagcgtgtcctccgccggcccaatcagccagtttaactacaagcaaagctttagcaaccctacctgcctgatcctggctacagtgcctcacaatctgacaacaatcacaaagcctctgaagtacagctacatcaacaagtgcagcagactgctgtccgatgacaggaccgaggtgccccagctggtgaacgccaatcagtacagcccatgcgtgagcattgtgcctagcacagtgtgggaggacggcgattactacagaaagcagctgagccccctggagggaggcggctggctggttgcatccggctccaccgtggccatgacagagcagctgcaaatgggcttcggaatcaccgtgcaatacggcacagatacaaactccgtgtgccccaagctggagtttgccaacgataccaagatcgcctcccagctgggcaactgagcggccgc

SEQ ID NO: 6 - protein, 492 aa, synthetic construct
MYRMQLLSCIALSLALVTNSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGN

SEQ ID NO: 7 - DNA, 756 bp, synthetic construct
  source 1..756: mol_type = other DNA
gaattcgccgccaccatgcctctgctgctgctgctccccctgctgtgggccggagctctggctagggtgcagcccaccgagagcatcgtgaggttccccaatatcacaaatctgtgtcccttcggcgaggtgtttaacgccaccaggtttgcctccgtgtacgcctggaataggaagagaatcagcaattgtgtggccgactacagcgtgctgtacaattccgccagcttctccaccttcaagtgctacggcgtgagccccaccaagctgaatgacctgtgttttaccaatgtgtacgccgacagcttcgtgatcaggggcgatgaggtgaggcagatcgcccccggccagacaggcaagatcgccgattacaattacaagctgcctgatgattttaccggctgtgtgatcgcctggaatagcaataacctggatagcaaggtgggcggcaactacaattacctgtacagactgtttagaaagtccaacctgaagcccttcgagagggacatcagcaccgagatctaccaggccggctccacaccttgtaacggcgtggagggcttcaactgctactttcccctgcagagctacggcttccagcccaccaatggcgtgggctaccagccttacagagtggtggtgctgagctttgagctgctgcacgcccccgccaccgtgtgtggacctaagaagagcaccaatctggtgaagaataaggccgtgaactttaactttaatggcctgtgaaagctt

SEQ ID NO: 8 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKAVNFNFNGL

SEQ ID NO: 9 - DNA, 756 bp, synthetic construct
  source 1..756: mol_type = other DNA
gaattcgccgccaccatgcctctgctgctgctgctccccctgctgtgggccggagctctggctagggtgcagcctacagagtccatcgtgaggtttcctaacatcacaaacctgtgtccttttggcgaggtgtttaatgccacaagatttgccagcgtgtacgcctggaataggaagaggatcagcaattgcgtggccgactactccgtgctgtacaatagcgccagcttttccacctttaagtgctacggcgtgagccccacaaagctgaatgacctgtgttttaccaacgtgtacgccgacagctttgtgatcaggggcgacgaggtgagacagatcgcccccggccagaccggcaatatcgccgattacaactacaagctgcctgacgatttcacaggctgcgtgatcgcctggaatagcaacaatctggacagcaaggtgggcggcaactacaattacctgtacaggctgttcagaaagtccaacctgaagccctttgagagggacatctccacagagatctaccaggccggctccaccccctgtaatggcgtgaagggctttaactgttactttcccctgcagagctacggcttccagcccacctacggcgtgggctaccagccctacagagtggtggtgctgagcttcgagctgctgcacgcccctgccaccgtgtgcggacctaagaagagcaccaacctggtgaagaacaaggccgtgaactttaactttaatggcctgtgaaagctt

SEQ ID NO: 10 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKAVNFNFNGL

SEQ ID NO: 11 - DNA, 756 bp, synthetic construct
  source 1..756: mol_type = other DNA
gaattcgccgccaccatgcctctgctgctgctgctccccctgctgtgggccggagctctggctagggtgcagcccaccgagagcatcgtgaggttccccaatatcacaaatctgtgtcccttcggcgaggtgtttaacgccaccaggtttgcctccgtgtacgcctggaataggaagagaatcagcaattgtgtggccgactacagcgtgctgtacaattccgccagcttctccaccttcaagtgctacggcgtgagccccaccaagctgaatgacctgtgttttaccaatgtgtacgccgacagcttcgtgatcaggggcgatgaggtgaggcagatcgcccccggccagacaggcaagatcgccgattacaattacaagctgcctgatgattttaccggctgtgtgatcgcctggaatagcaataacctggatagcaaggtgggcggcaactacaattacagatacagactgtttagaaagtccaacctgaagcccttcgagagggacatcagcaccgagatctaccaggccggctccaagccttgtaacggcgtggagggcttcaactgctactttcccctgcagagctacggcttccagcccaccaatggcgtgggctaccagccttacagagtggtggtgctgagctttgagctgctgcacgcccccgccaccgtgtgtggacctaagaagagcaccaatctggtgaagaataaggccgtgaactttaactttaatggcctgtgaaagctt

SEQ ID NO: 12 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKAVNFNFNGL

SEQ ID NO: 13 - protein, 13 aa, synthetic construct
AKFVAAWTLKAAA

SEQ ID NO: 14 - protein, 9 aa, synthetic construct
NVNFNFNGL

SEQ ID NO: 15 - protein, 457 aa, synthetic construct
MGWSCIILFLVATATGVHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 16 - protein, 438 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 17 - DNA, 1400 bp, synthetic construct
  source 1..1400: mol_type = other DNA
ggcgcgccgccgccaccatgggctggtcctgcatcatcctgtttctggtggctaccgtaccggcgtgcactctagagtgcagcctaccgagtctatcgtgcggttccccaacacaccaacctgtgtcctttcggcgaggtgttcaacgccaccagattcgcctctgtgtacgcctggaaccggaagcggatctctaactgcgtggccgactactccgtgctgtacaactccgcctccttcagcaccttcaagtgctacggcgtgtcccctaccaagctgaacgacctgtgcttcaccaacgtgtacgccgactccttcgtgatcagaggcgacgaagtgcggcagatcgctcctggacagaccggcaagatcgccgactacaactacaagctgcccgacgacttcaccggctgtgtgatcgcttggaactccaacaacctggactccaaagtcggcggcaactacaattaccggtacagactgttccggaagtccaacctgaagcctttcgagcgggacatctccaccgagatctaccaggctggctccaagccttgcaatggcgtggaaggcttcaactgctacttcccactgcagtcctacggcttccagcctacaaacggcgtgggctaccagccttacagagtggtggtgctgtccttcgagctgctgcatgctcctgctaccgtgtgcggccctaagaaatctaccaacctggtcaagaacaagcgggtgcagcccactgagagcattgtgcgcttccctaatatcacaaatctgtgcccctttgacgaagtctttaatgctacccgcttcgcttccgtgtatgcttggaatagaaagcggatcagcaattgcgtcgccgattacagcgtcctgtacaatctggccccattcttcacctttaagtgttatggcgtcagccccaccaagctcaatgatctctgttttaccaatgtctacgccgatagctttgtgattcgcggagatgaagtccgccagatcgcaccaggccagactggaaatatcgctgattacaattataagctccctgatgatttcacaggatgcgttatcgcctggaatagcaacaagctggacagcaaggtgtccggcaattacaactacctgtatcggctgttccgcaagagtaatctgaagccctttgagagagacatcagtacagaaatctatcaggccggcaacaagccctgtaacggcgtcgcaggctttaactgttattttcccctgcgctcctactccttccggcctacttatggcgtgggccatcaaccatatcgcgtggtggttctgagtttcgaactcctgcacgccccagccacagtgtgtggccccaaaaagagcaccaatctcgttaagaacaagtagtgaggccggcc

SEQ ID NO: 18 - DNA, 731 bp, synthetic construct
  source 1..731: mol_type = other DNA
gaattcgccgccaccatgcccctgctgctgctgctccctctgctgtgggccggcgctctggctagagtgcagcctacagagagcatcgtgaggttccctaatatcacaaacctgtgcccttttgacgaggtgttcaacgccacaaggtttgcctccgtgtacgcctggaacagaaagagaatcagcaattgtgtggccgattacagcgtgctgtacaatctggccccctttttcacatttaagtgttacggcgtgtcccccaccaagctgaatgatctgtgcttcaccaacgtgtacgccgacagctttgtgatcagaggcgacgaggtgagacagatcgcccctggccagaccggcaacatcgccgattacaactacaagctgcccgatgactttaccggctgcgtgatcgcctggaactccaacaagctggacagcaaggtgtccggcaactacaactacctgtacaggctgttcaggaagtccaatctgaagcctttcgagagagatatctccacagagatctaccaggccggcaacaagccctgcaatggcgtggccggctttaattgttactttcctctgcgaagctactcctttagacctacctacggcgtgggccaccagccttacagagtggtggtgctgtcctttgagctgctgcacgcccctgccacagtgtgtggccccaagaagtccaccaacctggtgaagaacaagtgagcggccgc

SEQ ID NO: 19 - protein, 235 aa, synthetic construct
MPLLLLLPLLWAGALARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 20 - protein, 472 aa, synthetic construct
EAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGN

SEQ ID NO: 21 - protein, 228 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKAVNFNFNGL

SEQ ID NO: 22 - protein, 15 aa, synthetic construct
GGGGSGGGGSGGGGS

SEQ ID NO: 23 - protein, 20 aa, synthetic construct
GGGGSGGGGSGGGGSGGGGS

SEQ ID NO: 24 - protein, 8 aa, synthetic construct
GSGSGSGS

SEQ ID NO: 25 - protein, 4 aa, synthetic construct
SGSG

SEQ ID NO: 26 - protein, 9 aa, synthetic construct
CGGSGSGSG

SEQ ID NO: 27 - protein, 4 aa, synthetic construct
GSGC

SEQ ID NO: 28 - protein, 4 aa, synthetic construct
GSGS

SEQ ID NO: 29 - protein, 4 aa, synthetic construct
GGGS